(19) United States
(12) Patent Application Publication
(10) Pub. No.: US 2023/0279061 A1
(43) Pub. Date: Sep. 7, 2023

(54) ISOLATED PEPTIDE FOR A PEPTIDE COACERVATE, AND METHODS OF USE THEREOF

(71) Applicant: NANYANG TECHNOLOGICAL UNIVERSITY, Singapore (SG)

(72) Inventors: Ali Gilles Tchenguise MISEREZ, Singapore (SG); Yue SUN, Singapore (SG)

(21) Appl. No.: 18/000,226

(22) PCT Filed: Jun. 1, 2021
(86) PCT No.: PCT/SG2021/050309

§ 371 Date: Nov. 29, 2022

(30) Foreign Application Priority Data
Jun. 1, 2020 (SG) 10202005129Q

(51) Int. Cl. C07K 14/435; A61K 9/50; A61K 47/68

(52) U.S. Cl. CPC C07K 14/43504; A61K 9/5052; A61K 47/68

(57) ABSTRACT

The present invention relates to an isolated peptide modified based on the histidine-rich beak peptide (HBpep), which is derived from the Humbolt squid beak protein. In a preferred embodiment, the isolated peptide comprises the amino acid sequence of GHGVYGHGVYGHGPYKGHGPYGHGLYW (SEQ ID NO: 10), which contains a single lysine residue inserted at position 16 from the N-terminal of HBpep. In a further preferred embodiment, the lysine residue is conjugated with a self-immolative moiety, preferably comprising a disulfide moiety. The present invention also relates to a composition for the delivery of an active agent, wherein the composition comprises a peptide coace rvate comprising the isolated peptide and the active agent recruited in the peptide coace rvate. The present invention further relates to a method of recruiting the active agent in the peptide coace rvate, a method of delivering the active agent, and a method of treating or diagnosing a condition or disease in a subject.

### CROSS REFERENCE TO RELEATED APPLICATIONS

This patent application claims priority to Singapore Application No. 10202005129Q entitled “Redox-Responsive Peptide Coacervates for Intra-Cellular Delivery of an Active Agent”, filed on 01 Jun. 2020, the disclosures of which is incorporated herein by reference in its entirety.

### TECHNICAL FIELD

The present invention lies in the field of targeted delivery of active agents using peptide coacervates including isolated peptides, methods of peptide coacervate formation, and active agent recruitment and delivery using the peptide coacervates.

### BACKGROUND

Biomacromolecules, including peptides (Jin, J. et al., Theranostics, 2020, 10, 10141; Zou, P. et al., Biomat. Sci., 2020, 8, 4975), proteins (Guillard, S. et al., Trends in Biotech., 2015, 33, 163; Fu, A. et al., Bioconjugate Chem., 2014, 25, 1602; Nelson, A.L. et al., Nat. Reviews Drug Disc., 2010, 9, 767) and RNAs (Dowdy, S.F. et al., Nat. Biotech., 2017, 35, 222; Jackson, L.A. et al. New Eng. J. Med., 2020, 383, 1920), offer promising therapeutic prospects for the treatment of various diseases owing to key advantages such as high potency, specificity, or safety (Du, S. et al., J. Am. Chem. Soc., 2018, 140, 15986). However, their full therapeutic potential has not been fulfilled because of their poor cell membrane permeability and/or endosomal trapping that limits their intracellular release (Goswami, R. et al., Trends in Pharmacol. Sci., 2020, 41, 743).

Current strategies to tackle such issues rely on nanoscale carriers such as inorganic nanoparticles (Scaletti, F. et al., Chem. Soc. Reviews, 2018, 47, 3421), synthetic polymers (Liu, C. et al. Sci. Adv, 2019, 5, eaaw8922) or nanoscale hybrid assemblies that can mediate cell membrane fusion (Mout, R. et al., ACS Nano., 2017,11, 2452; Lee, S. et al., J. Am. Chem. Soc., 2020, 142, 12157). In alternative approaches, the macromolecular drugs are conjugated or complexed with cell-penetrating peptides (Li, M. et al., J. Am. Chem. Soc., 2015, 137, 14084; Akishiba, M. et al., Nat. Chem., 2017, 9, 751) that enhance endosomal escape. While these methods are promising and increasingly considered for clinical translation, they also exhibit pitfalls (Du, S. et al., J. Am. Chem. Soc., 2018, 140, 15986). The fabrication methods can be complex and may involve the use of organic solvents that can affect the bioactivity of cargo biomacromolecules (Hu, Y. et al., Chem. Soc. Reviews, 2018, 47, 1874; Buse, J. et al., Nanomed., 2010, 5, 1237). Further, some carriers are limited to a specific type of biomacromolecules, whereas in some cases the release is restricted to relatively small molecular weight cutoffs (Yang, J et al., Adv Healthcare Mat., 2017, 6, 1700759; Tai, W. et al., Sci. Adv., 2020, 6, eabb0310). Safety concerns have also been raised for some carriers like inorganic and lipid nanoparticles (Buse, J. et al., Nanomed., 2010, 5, 1237; Khlebtsov, N. et al., Chem. Soc. Reviews, 201 1, 40, 1647; Fadeel, B. et al., Adv. Drug Delivery Reviews, 2010, 62, 362). Whether the carriers are inorganic or organic-based (polymers, lipids, peptides or fusions thereof), it is also generally considered that they must remain below ca. 200 nm to cross the cell membrane (Goswami, R. et al., Trends in Pharmacol. Sci., 2020, 41, 743; Yang, J et al., Adv Healthcare Mat., 2017, 6, 1700759).

Thus, there is a need to develop safe delivery platforms that can cross the cell membrane, are not trapped inside endosomal vesicles to directly deliver the biomacromolecules. Further, the need to remain below ca. 200 nm to cross the cell membrane adds to the challenge of designing such platforms for larger biomacromolecules. In addition, it is desired that the recruitment method does not affect the bioactivity of the biomacromolecule and that the carriers exhibit negligible cytotoxicity.

Coacervation or liquid-liquid phase separation (LLPS) refers to the de-mixing of a homogenous polymer solution into two distinct phases: a concentrated macromolecule-rich (or coacervate) phase and a dilute macromolecule-depleted phase. An example of a biomacromolecules which exhibit coacervation (or LLPS) properties include the histidine-rich beak peptide (HBpep). HBpep is derived from the Humbolt squid beak protein and its self-coacervation property plays an essential role in the formation of the mechanical gradient of squid beaks (Tan et al., Nat. Chem. Biol., 2015, 11 (7), 488). HBpep is characterized by a low sequence complexity consisting of only 5 copies of the tandem repeat GHGXY (where X could be leucine (L), proline (P), or valine (V)) and a single C-terminal Trp (W) residue. Further, a key feature of the HBpep is the presence of 5 His (H) residues in the 5 repeat sequence motifs GHGXY that confer pH-responsivity LLPS behavior (Gabryelczyk, B. et al., Nat. Comms., 2019 10, 5465). Notably, this allows the HBpep to remain in a monomeric state at a low pH, but to quickly phase separate or self-coacervate into coacervate microdroplets at neutral pH and to concomitantly recruit various macromolecules from the solution during the process.

A previous study by the inventors has shown that HBpep coacervates have the ability to recruit various biomacromolecules with high efficiency of above 95%, and exhibit low toxicity (Lim, Z.W. et al., Bioconjugate Chem., 2018, 29, 2176). HBpep coacervates were also recently demonstrated to be able to cross the cell membrane via an endocytosis-free pathway (Lim, Z.W. et al., Acta Biomat., 2020, 110, 221). It has therefore been suggested that self-coacervating HBpeps may be potential candidates for intracellular delivery of therapeutics. Preliminary attempts to use HBpep coacervates to recruit and deliver proteins resulted in successful transmembrane delivery. For example, the inventors observed that HBpep coacervates successfully recruited biomacromolecules such as insulin and doxorubicin, and delivered said coacervates intracellularly (US 2019/0388357 A1). However, said strategy had the drawback that the HBpep microdroplets formed organelle-like structures within the cells and did not readily release their cargos.

Therefore, there still exists a need for a novel and safe delivery platform for both the intracellular delivery and direct cytosolic release of a large variety of biomacromolecular therapeutics. Such platforms would have promising potential in the treatment of cancers, metabolic diseases, or as vaccines.

### SUMMARY

The inventors have found that the previously existing drawbacks of delivery platforms based on HBpep coacervates could be overcome by using modified peptides, as described herein, for coacervate formation. Thus, the present invention is based on the inventors’ finding that peptide coacervates formed from the (modified) isolated peptides described herein can be used for the efficient delivery and intracellular release of active agents. The isolated peptide coacervates formed may co-recruit one, two or more active agents to be applicable and effective in the management and/or treatment of diseases or disorders, such as cancer. Additionally, the inventors’ findings provide general guidelines and concepts for designing isolated peptide coacervates with LLPS ability for direct cytosolic release of the active agents which may be applicable in various applications, including bio-inspired protocells and smart drug-delivery systems.

In a first aspect, the present invention is thus directed to an isolated peptide including the amino acid sequence
(GHGXY)ₙ K (GHGXY)ₘ Z,
(GHGXY K)ₙ (GHGXY)ₘ Z, or
(GHGXY)ₙ (K GHGXY)ₘ Z, wherein
X is valine (V), leucine (L) or proline (P),
Z is tryptophan (W) or absent,
n is 0, 1, 2, 3, 4 or 5,
m is 0, 1, 2, 3, 4 or 5,
n+m is 3, 4 or 5, preferably 5.

Non-limiting isolated peptides comprise or consists of an amino acid sequence, such as but not limited to:
(i) K GHGXY GHGXY GHGXY GHGXY GHGXY W (SEQ ID NO: 1)
(ii) GHGXY K GHGXY GHGXY GHGXY GHGXY W (SEQ ID NO: 2)
(iii) GHGXY GHGXY K GHGXY GHGXY GHGXY W (SEQ ID NO: 3)
(iv) GHGXY GHGXY GHGXY K GHGXY GHGXY W (SEQ ID NO: 4)
(v) GHGXY GHGXY GHGXY GHGXY K GHGXY W (SEQ ID NO: 5)
(vi) GHGXY GHGXY GHGXY GHGXY GHGXY W K (SEQ ID NO: 6)
(vii) K GHGVY GHGVY GHGPY GHGPY GHGLY W (SEQ ID NO: 7)
(viii) GHGVY K GHGVY GHGPY GHGPY GHGLY W (SEQ ID NO: 8)
(ix) GHGVY GHGVY K GHGPY GHGPY GHGLY W (SEQ ID NO: 9)
(x) GHGVY GHGVY GHGPY K GHGPY GHGLY W (SEQ ID NO: 10)
(xi) GHGVY GHGVY GHGPY GHGPY K GHGLY W (SEQ ID NO: 11), or
(xii) GHGVY GHGVY GHGPY GHGPY GHGLY W K (SEQ ID NO: 12)

In various embodiments, the lysine residue (K) is modified at an epsilon (ε)- amino group with a self-immolative moiety.

In various embodiments, the self-immolative moiety comprises a disulfide (—S—S—) moiety.

In various non-limiting embodiments, the self-immolative moiety has the formula such as but not limited to: —C(═O)—O—(CH₂)ₙ—S—S—R, wherein R is selected from: substituted or unsubstituted alkyl, alkenyl, cycloalk(en)yl, and aryl, and n is an integer from 1 to 10, for example 1, 2, 3, 4, or 5.

In various non-limiting embodiments, R may be a group of the formula such as but not limited to: —(CH₂)ₙ—O—C(═O)—R′, wherein n is 1, 2, 3, 4, or 5, and wherein R′ is selected from: C1-C4 alkyl, aryl, preferably phenyl, said alkyl or aryl group optionally substituted with halogen.

In another aspect, the present invention is directed to a composition for the delivery of an active agent that comprises a peptide coacervate, which comprises or consists the one or more (isolated) peptides of the invention, and an active agent recruited in the peptide coacervate.

In various embodiments, the self-immolative moiety of the peptide coacervate autocatalytically cleaves itself upon exposure to specific conditions selected from the group such as but not limited to: pH changes, redox changes, exposure to release agents, and combinations thereof. In some embodiments, the release agent is glutathione (GSH), specifically, cell endogenous GSH, which is ubiquitous in cells.

In various embodiments, the active agent includes, but is not limited to proteins, (poly)peptides, carbohydrates, nucleic acids, lipids, chemical compounds, nanoparticles, antibodies, and combinations thereof.

In various embodiments, the active agent is a pharmaceutical or diagnostic agent.

In various embodiments, the pharmaceutical or diagnostic agent is a (macro)molecular therapeutic agent, for example an anti-cancer agent. In some embodiments, the anti-cancer agent may include or be, but is not limited to, agent(s) such as saporin, second mitochondria-derived activator of caspases peptide (Smac), proapoptotic domain peptide (PAD), either alone or in combinations thereof. In some embodiments, the pharmaceutical or diagnostic agent is lysozyme, bovine serum albumin (BSA), phycoerythrin (R-PE), enhanced green fluorescence protein (EGFP), β-galactosidase (β-Gal), either alone or in combinations thereof. In some other embodiments, the pharmaceutical or diagnostic agent is luciferase-encoding mRNA, EGFP-encoding mRNA, either alone or in combinations thereof. The pharmaceutical and diagnostic agents specifically disclosed herein serve as proof-of-concept that a variety of different molecules and in particular a broad variety of polypeptides with different molecular weights and isoelectric points can be successfully recruited. It is thus understood that while certain embodiments of the invention are directed to these exemplified embodiments, the present disclosure is not limited thereto. In particular, the skilled person would be aware that these data serve as proof-of-concept and that the inventive concept can be extended to various alternative agents.

In various embodiments, the composition is a pharmaceutical or diagnostic formulation for administration to a subject. In various embodiments, it can thus comprise any one or more auxiliaries, carriers and excipients that are pharmaceutically or diagnostically acceptable. In some embodiments, the composition is a liquid. The subject may be a mammal, for example, a human being.

In various embodiments, the pH of the composition is 5.0 or higher, for example, in the range of 5.5 to 8.0.

In still another aspect, the present invention relates to a method for the recruitment of an active agent in a peptide coacervate, the method comprising: (1) providing an aqueous solution of coacervate-forming peptides, said coacervate-forming peptides comprising one or more isolated peptides of the invention, (2) combining the aqueous solution of the coacervate-forming peptides with an aqueous solution of an active agent, and (3) inducing coacervate formation.

In various embodiments, the active agents in the combined aqueous solution are also provided in the form of an aqueous solution. Said aqueous solution may have a pH below 8.0, and in some embodiments, is buffered such that the combination of the aqueous solution of the active agent with the aqueous solution of the coacervate-forming peptides obtained in the combined aqueous solution has a pH below 8.0, for example, in the range of 5.5 to 7.5. In some embodiments, coacervate formation is facilitated when the combination of the aqueous solution with the active agent and the combination of the coacervate-forming peptides is between pH 5.5 to 7.0. For example, coacervate forming may be induced at pH below 7.0, for example, at 6.5 or at 6.0.

In various embodiments, a volume ratio of the aqueous solution of the aqueous solution of the coacervate-forming peptides to the aqueous solution of the active agent may be greater than 1: 5, for example, in the range of 1 : 5 to 1 : 20. In some embodiments, the volume ratio of the aqueous solution of the aqueous solution of the coacervate-forming peptides to the aqueous solution of the active agent is between 1 : 8 to 1 : 10, for example, at about 1 : 9, or at about 1 : 9.5.

In a still further aspect, the present invention is directed to a method for the delivery of an active agent, said method comprising: (1) providing a composition including a peptide coacervate that comprises one or more isolated peptides of the invention, and an active agent recruited in the peptide coacervate, (2) exposing the peptide coacervate to conditions that trigger the release of said active agent from the peptide coacervate. The conditions that trigger the release of the active agent may be selected from those disclosed above for the composition for the delivery of the active agent.

In still another aspect, the invention further encompasses a method for treating or diagnosing a condition or disease in a subject in need thereof, said method comprising: (1) administering a composition according to the invention, i.e. a composition including a peptide coacervate as described herein, to a subject. The peptide coacervate includes one or more isolated peptides of the invention, and a pharmaceutical or diagnostic agent recruited in the peptide coacervate, and (2) exposing the peptide coacervate to conditions that trigger the release of said pharmaceutical or diagnostic agent from the peptide coacervate. The conditions that trigger the release of the pharmaceutical or diagnostic agent may be selected from those disclosed above for the composition for the delivery of the pharmaceutical or diagnostic agent. The subject may be a mammal, for example, a human being.

In further exemplary embodiments, the subject is a human afflicted by cancer, and the pharmaceutical or diagnostic agent is a macromolecular therapeutic agent, for example a protein and/or peptide-based therapeutic agent. In some embodiments, the pharmaceutical or diagnostic agent is an anti-cancer agent, such as saporin, second mitochondria-derived activator of caspases peptide (Smac), proapoptotic domain peptide (PAD), either alone or in combinations thereof. The release of the pharmaceutical or diagnostic agent is facilitated by the exposure of the peptide coacervate to GSH, i.e. cell endogenous GSH, present in the cytosol of cells and the resulting reduction of the disulfide bond of the peptide coacervate.

The details of one or more embodiments of the disclosure are set forth in the accompanying drawings and the description below. Other features, objects and advantages will be apparent from the description and drawings, and from the claims.

### BRIEF DESCRIPTION OF THE DRAWINGS

FIG. 1. Schematic illustration of the design of redox-responsive peptide coacervates HBpep-SR with direct cytosolic entry that bypasses endocytosis. HBpep-K (top left) remains in solution at neutral pH but can phase separate and form coacervates after conjugation of the sole lysine residue (K) with a self-immolative moiety (HBpep-SR, middle left). In a reducing environment such as the glutathione (GSH)-rich cytosol, HBpep-SR is reduced, followed by self-catalytic cleavage of the SR moiety, resulting in HBpep-K again and in the disassembly of the coacervates (left bottom). During coacervation of HBpep-SR near neutral pH (top right), macromolecular therapeutics are readily recruited within the coacervates. Upon incubation with cells, the therapeutics-loaded coacervates cross the cell membrane to migrate directly in the cytosol (right bottom), whereupon they are reduced by GSH resulting in the disassembly and release of the therapeutic.

FIG. 2. Synthesis routes to produce the self-immolative (SR) moieties that are subsequently conjugated to HBpep-K. (A) Synthesis and coupling of intermediate products HO—SS—R and N-hydroxysuccinimide (NHS). The end group of the moiety is (B) acetate (labelled as “SA” below); and (C) benzoate (labelled as “SP” below).

FIG. 3. ¹H NMR spectra of synthesized products in CDCl₃. (A) HO—SS—Ac; (B) NHS—SS—Ac; (C) HO—SS—Ph; and (D) NHS—SS—Ph.

FIG. 4. MALDI-TOF mass spectra of HBpep and HBpep conjugated peptides. (A) Fmoc-HBpep-K (theoretical MW: 3132.4 Da); (B) HBpep-SA (theoretical MW: 3132.4 Da); and (C) HBpep-SP (theoretical MW: 3194.5 Da).

FIG. 5. Characterization of modified HBpep coacervates. (A) Turbidity measurements of HBpep-SA and HBpep-SP at various pH and comparison with HBpep-K. (B) Optical micrograph of HBpep-SP coacervates at pH 6.5 and ionic strength 0.1 M (phosphate buffer). (C) Particle size of pristine, EGFP-loaded, and mRNA-loaded coacervates. (D) Fluorescence micrograph of EGFP-loaded HBpep-SP coacervates. (E) Fluorescence micrograph of Cy5-mRNA-loaded HBpep-SP coacervates. Data are presented as the mean ± SD of n = 3 independent measurements.

FIG. 6. Characterization of modified HBpep coacervates in the presence of reducing agents. Dithiothreitol (DTT)-induced reduction of (A) HBpep-SA coacervates; (B) HBpep-SP coacervates; and (C) GSH-induced reduction of HBpep-SA and HBpep-SP coacervates. Data are presented as the mean ± SD of n = 3 independent measurements.

FIG. 7. Intracellular delivery of EGFP and insulin. Control (A) fluorescence; and (B) brightfield micrographs of HepG2 cells treated with free EGFP. (C) 4 hours and (D) 24 hours fluorescence micrographs of HepG2 cells treated with EGFP-loaded HBpep-SA coacervates for 24 hours. (E) 4 hours and (F) 24 hours fluorescence micrographs of HepG2 cells treated with EGFP-loaded HBpep-SP coacervate. (G) 4 hours and (H) 24 hours fluorescence micrographs of HepG2 cells treated with FITC-insulin-loaded HBpep-SA coacervates. (I) 4 hours and (J) 24 hours fluorescence micrographs of HepG2 cells treated with FITC-insulin-loaded HBpep-SP coacervates. (K-L) Intracellular delivery of EGFP into A549 (K), NIH 3T3 (L), and HEK293 (M) cells from HBpep-SA coacervates.

FIG. 8. Intracellular protein delivery into HepG2 cells. (A) Summary of proteins with a wide range of isoelectric point (IEP) and molecular weight (MW) demonstrated to be successfully delivered in the cytosol, including lysozyme (IEP: 10.7; MW: 14 kDa), saporin (IEP: 9.4; MW: 28.6 kDa), bovine serine albumin (BSA; IEP: 4.8; MW: 66.4 kDa), R-phycoerythrin (R-PE; IEP: 4.1; MW: 255 kDa); and β-galactosidase (β-Gal; IEP: 4.6; MW: 465 KDa). (B) Recruitment efficiency of proteins by HBpep-SP coacervates (1 mg/mL), including EGFP, AF-lysozyme, AF-BSA and R-PE (0.1 mg/mL). (C) AF-lysozyme delivery mediated by HBpep-SP coacervates. (D) AF-BSA delivery mediated by HBpep-SP coacervates. Control (E) fluorescence; and (F) brightfield micrographs of HepG2 cells treated with free AF-lysozyme for 24 hours. Control (G) fluorescence; and (H) brightfield micrographs of HepG2 cells treated with free AF-BSA. (I) R-PE delivery mediated by HBpep-SP coacervates for 24 hours. Control (J) fluorescence; and (K) brightfield micrographs of HepG2 cells treated with free R-PE for 24 hours. (L-N) Co-delivery of EGFP and R-PE by HBpep-SP coacervates. (L) EGFP channel; (M) R-PE channel; and (N) merged micrographs of HepG2 cells treated with EGFP / R-PE co-loaded HBpep-SP coacervates for 24 hours. (O) Concentration-dependent cytotoxicity of free saporin and saporin-loaded HBpep-SP coacervates. (P) X-Gal staining of cells treated with β-Gal-loaded HBpep-SP coacervates after 24 hours. (Q) X-Gal staining of cells treated with free β-Gal-comparing. Data are presented as the mean ± SD of n = 3 independent measurements.

FIG. 9. Intracellular peptide delivery into HepG2 cells. (A-B) FITC-Smac delivery mediated by HBpep-SP coacervates (A) and comparison with free FITC-Smac (B). (C) Concentration-dependent cytotoxicity of Smac and Smac-loaded HBpep-SP coacervates. (D-E) FITC-PAD delivery mediated by HBpep-SP coacervates (D) and comparison with free FITC-PAD (E). (F) Concentration-dependent cytotoxicity of PAD and PAD-loaded HBpep-SP coacervates. Data are presented as the mean ± SD of n = 3 independent measurements.

FIG. 10. Intracellular mRNA transfection and cytotoxicity of redox-responsive coacervates. (A-B) Luciferase-encoding mRNA transfection efficiency of HBpep-SA and HBpep-SP coacervates compared to common commercial transfection reagents including PEI and lipofectamine 2000 and 3000 in HepG2 cells (A); and HEK293 cells (B). (C-D) Relative cell viability of HepG2 cells (C); and HEK293 cells (D) treated with HBpep-SA and HBpep-SP coacervates and comparison with commercial transfection reagents including PEl and lipofectamine 2000 and 3000. (E-F) Fluorescence micrograph of luciferase-encoding mRNA transfection of HBpep-SA and HBpep-SP coacervates in HepG2 cells (E); and HEK293 cells (F). (G) FACS of HepG2 cells transfected with EGFP-encoding mRNA (Cy5 labeled) loaded in HBpep-SP coacervates; and (H) FACS of untreated HepG2, i.e. control group. (I) FACS of HEK293 cells transfected with EGFP-encoding mRNA (Cy5 labeled) loaded in HBpep-SP coacervates; and (J) FACS of untreated HEK293, i.e. control group. Data are presented as the mean ± SD of n = 3 independent measurements.

FIG. 11. Cell internalization study of coacervates. (A) Confocal microscopy image of HepG2 cells treated with EGFP-loaded HBpep-SP coacervates (green) for 2 hours. The nucleus was stained with Hoechst (blue) and the lysosomes were stained with LysoTracker (red). Coacervates are not co-localized with lysosomes. (B) FACS and (C) fluorescence micrographs of HepG2 cells treated with various inhibitors before incubation with EGFP-loaded HBpep-SP coacervates for 4 hours. MβCD: methyl-β-cyclodextrin; NaN3— sodium azide; AM: amiloride; CPM: chlorpromazine. Only the cholesterol-depletion compound MβCD inhibits cell uptake. Data are presented as the mean ± SD of n = 3 independent measurements.

### DETAILED DESCRIPTION

The inventors’ found that engineered artificial peptides derived from histidine-rich beak peptide (HBpep) that additionally comprise a lysine residue (K) between the pentapeptide repeats or at the termini of such a peptide provide for a means to overcome the previous drawback of delayed or impaired intracellular release of the cargo from a coacervate formed by these peptides. Specifically, the inventors have found that the coacervates formed from such engineered peptides and are stimuli-responsive in that they disassemble and thus release the cargo once exposed to the reducing environment and physiological pH of a cell’s cytosol.

In a first aspect, the present invention is thus directed to such modified peptides (HBpep-K), preferably in isolated form, that comprise, consist essentially of or consist of the amino acid sequence
(GHGXY)ₙ K (GHGXY)ₘ Z,
(GHGXY K)ₙ (GHGXY)ₘ Z, or
(GHGXY)ₙ (K GHGXY)ₘ Z, wherein
X is valine (V), leucine (L) or proline (P),
Z is tryptophan (W) or absent,
n is 0, 1, 2, 3, 4 or 5,
m is 0, 1, 2, 3, 4 or 5, and
n+m is 3, 4 or 5, preferably 5.

In various embodiments, the isolated peptides (HBpep-K) comprise, consist essentially of or consist of the amino acid sequence (GHGXY)ₙ K (GHGXY)ₘ Z, i.e. comprise only a single K residue in the indicated consensus sequence.

In the above sequence and all further sequences disclosed below, amino acids are identified by their one letter code, Thus, G stands for glycine, H stands for histidine, L stands for leucine, Y stands for tyrosine, K stands for lysine, etc. The isolated peptides (HBpep-K) are also shown in the conventional manner, i.e. in the N- to C-terminal orientation. The individual amino acids are covalently coupled to each other by peptide bonds. If an amino acid is not defined or defined as being “any amino acid”, this typically refers to the 20 naturally occurring proteinogenic amino acids G, A, V, L, I, F, W, Y, S, T, P, C, M, D, E, N, Q, K, H, and R.

The term “peptide”, as used herein, relates to polymers of amino acids, typically short strings of amino acids. In various non-limiting embodiments, the peptides may include only amino acids selected from the 20 proteinogenic amino acids encoded by the genetic code, namely, glycine, alanine, valine, leucine, isoleucine, phenylalanine, proline, serine, threonine, asparagine, glutamine, tyrosine, tryptophan, histidine, arginine, lysine, aspartic acid, glutamic acid, cysteine, and methionine. These amino acids are also designated herein by their three or one letter code (as above). Generally, peptides may be dipeptides, tripeptides or oligopeptides of at least 4 amino acids in length. The typical length for the peptides of the invention may range from at least about 16 amino acids to 100, preferably to 80, 70, 60 or 50 amino acids in length, for example, at least 16, 17, 18, 19, 20, 21, 22, 23, 24, 25, 26, 27, 28, 29, or 30 amino acids in length, the upper limit for example, being 50, 40 or 35 amino acids. Generally, it may be preferred to use peptides as short as possible without impairing their functionality. Accordingly, the term “peptide(s)”, as used herein, refers to a unique polymer of amino acids, in accordance with various embodiments.

The term “isolated”, as used herein, relates to the fact that the referenced peptide is at least partially separated from other components it may (naturally or non-naturally) associate with, for example other molecules, cellular components and cellular debris. Said isolation may be achieved by purification protocols for proteins and peptides well known to those skilled in the art.

The term “protein”, as used herein, relates to polypeptides, i.e. polymers of amino acids connected by peptide bonds, including proteins that comprise multiple polypeptide chains. A polypeptide typically comprises more than 50, for example, 100 amino acids or more.

The term “(amino acid) residue”, as used herein, relates to one or more amino acids which are considered as part of the peptide.

The term “about”, as used herein, in connection with a numerical value, means said value ± 10 %, for example, ± 5 %.

In the above, the isolated peptides (HBpep-K) has a minimum length of 16 amino acids, for example 17 amino acids, and comprise at least three sequence motifs, GHGXY, K and optionally Z. For example, the sequence motif may include at least one sequence motif GHGVY, at least one sequence motif GHGPY, and one sequence motif GHGLY. As a further example, the isolated peptides (HBpep-K) may include at least four copies, or may include five copies of the sequence motif GHGXY, Z, and K. In various embodiments, the isolated peptides (HBpep-K) may include, for example, two copies of the sequence motif GHGVY, two copies of the sequence motif GHGPY and one copy of the sequence motif GHGLY. The C-terminal amino acid, Z, which may represent tryptophan (Trp or W), may be present or may be absent.

The isolated peptides (HBpep-K) may consist of the given amino acid sequence. In such embodiments, there are no further N- and/or C-terminal flanking peptide sequences. Alternatively, the isolated peptides (HBpep-K) may essentially consist of the amino acid sequence given. In such embodiments, there may be N- and/or C-terminal peptide sequences that flank the core consensus sequence. These are in such embodiments 1 to 10 amino acids in length, for example 1, 2, 3, 4, 5, 6, 7, 8, 9 or 10 amino acids in length. In such embodiments, it may be preferred that the flanking sequences in sum are not longer than the core sequence defined by the above consensus sequence. Finally, the isolated peptides (HBpep-K) may comprise the amino acid sequence. In such embodiments, the flanking sequences may be longer than 10 amino acids, for example up to 30 amino acids, and may, in sum, be longer than the conserved core motif. The flanking sequences may comprise further motifs GHGXY and further K residues, if desired. However, in various embodiments, they do not comprise any further GHGXY motif. In various embodiments, it is preferred that the peptides of the invention consist of or consist essentially of the sequence given herein. It is generally advantageous to use a peptide that only includes the minimum sequence necessary to fulfil its function, i.e. in the present case form a coacervate and disassemble under the desired conditions.

The upper limit in peptide length of the isolated peptides (HBpep-K) may be 50 amino acids, for example, up to 40, up to 35 or up to 30 amino acids. In various embodiments, the isolated peptides (HBpep-K) may be 27 amino acids, including five copies of the tandem repeat if the sequence motif GHGXY (i.e., n + m = 5), Z and the K residue. In various embodiments, it is preferred that the isolated peptides (HBpep-K) comprise no more than five sequence motifs, GHGXY, Z and the K residue, and therefore comprise no more than 27 amino acids, i.e. have a maximum length of 27 amino acids.

In various embodiments, the isolated peptides (HBpep-K) which includes the amino acid sequence described above, may be histidine-rich proteins.

The term “histidine-rich proteins”, as used herein, relates to proteins that include at least three histidine residues and overall, have a comparably high amount of residues of the amino acid histidine (His or H). This may mean that the histidine content of a given protein is above 3%, for example, greater than 5% or greater than 10%, or greater than 12%, or greater than 14%, or greater than 16%, or greater than 17%, or greater than 18%, relative to the total number of amino acids in the peptide sequence.

As the isolated peptides (HBpep-K) are variants of histidine-rich proteins that do not occur in nature and have typically been artificially produced, the isolated peptides (HBpep-K) are, in various embodiments, artificial peptides, such as those created by genetic engineering techniques, recombinant peptides and the like known to those skilled in the art.

In various non-limiting embodiments, the isolated peptides (HBpep-K) of the above comprise, consist essentially of or consist of an amino acid sequence selected from the group consisting of:
K GHGXY GHGXY GHGXY GHGXY GHGXY W (SEQ ID NO: 1)
GHGXY K GHGXY GHGXY GHGXY GHGXY W (SEQ ID NO: 2)
GHGXY GHGXY K GHGXY GHGXY GHGXY W (SEQ ID NO: 3)
GHGXY GHGXY GHGXY K GHGXY GHGXY W (SEQ ID NO: 4)
GHGXY GHGXY GHGXY GHGXY K GHGXY W (SEQ ID NO: 5)
GHGXY GHGXY GHGXY GHGXY GHGXY W K (SEQ ID NO: 6)
K GHGVY GHGVY GHGPY GHGPY GHGLY W (SEQ ID NO: 7)
GHGVY K GHGVY GHGPY GHGPY GHGLY W (SEQ ID NO: 8)
GHGVY GHGVY K GHGPY GHGPY GHGLY W (SEQ ID NO: 9)
GHGVY GHGVY GHGPY K GHGPY GHGLY W (SEQ ID NO: 10)
GHGVY GHGVY GHGPY GHGPY K GHGLY W (SEQ ID NO: 11), or
GHGVY GHGVY GHGPY GHGPY GHGLY W K (SEQ ID NO: 12)

All the above isolated peptides (HBpep-K) sequences may include additionally N-and/or C-terminal amino acids, i.e. flanking sequences as have been defined above. The C-terminal tryptophan (W) may be absent or present. In various embodiments, the isolated peptides (HBpep-K) has a maximum length of 30 amino acids, for example 28 amino acids or less, or 27 amino acids or less.

In various embodiments, the isolated peptides (HBpep-K) may be synthesized using any conventional peptide synthesis method, including chemical synthesis and recombinant production, for example, solid phase peptide synthesis. Suitable methods are well-known to those skilled in the art and may be selected using their routine knowledge.

It has been found that the isolated peptides (HBpep-K) comprising the artificially introduced single lysine residue (K) exhibit altered coacervation and recruitment properties, as compared to histidine-rich peptides which do not comprise the lysine residue (HBpep). In particular, it has been observed that the isolated peptides of the invention (HBpep-K) form coacervates, i.e. phase separate, at an increased pH of 9.0 (as opposed to peptides which do not include the lysine residue (K) which form coacervates under neutral conditions). At near neutral conditions, i.e. pH of about 5.0 to 8.0, the isolated peptides of the invention (HBpep-K) remain as monomeric peptides in solution. This changed properties are due to lysine (K) being a positively charged amino acid, with the inclusion of said lysine residue (K) in the isolated peptides (HBpep-K) shifting the isoelectric point and increasing the hydrophilicity of the unmodified peptide (HBpep), which in turn affects the phase separation behaviour of said isolated peptide (HBpep-K). This changed behaviour allows tuning of the coacervate formation/disassembly properties, as will be detailed below.

In various embodiments, the lysine residue (K) of the isolated peptides (HBpep-K) is modified, at the epsilon (ε)- amino group with a self-immolative (SR) moiety. The ε-amino group of the lysine residue (K) is side chain amino group, is nucleophilic and thus provides for a highly reactive group that can serve as a reaction site for the modification of the lysine residue (K). The conjugation of the lysine residue (K) to the self-immolative (SR) moiety produces the modified isolated peptides (HBpep-SR), referred herein after as “modified isolated peptides (HBpep-SR)”. Said modification of the lysine residue (K) can be used to tune the coacervate formation and disassembly properties, as it may be used to mask the charge of the lysine residue (K) under neutral conditions and thus influence phase separation behavior that is dependent on the charge properties of the peptide.

The term “self-immolative (SR) moiety”, as used herein, refers to a moiety that is self-cleaving upon encountering a certain triggering stimulus, such as a change in pH or redox potential. “Self-immolative” and “self-cleaving” are thus used interchangeably herein. In response to such a stimulus, the molecule autocatalytically cleaves itself to release the functional group, typically in form of a harmless by-product, such that the unmodified side chain amino group of the lysine residue (K) is reformed.

In various embodiments, the self-immolative modification is a modification by an organic moiety. Said modification may serve to adjust phase separation behavior, for example by masking the charge of the lysine residue (K) and/or increasing hydrophobicity. In various embodiments, the modification refers to the conjugation of the self-immolative (SR) moiety at the ε- amino group of the lysine residue (K). For example, the self-immolative (SR) moiety may be conjugated to the amine, i.e. NH₂ group of the lysine residue (K), in other words, conjugated to the ε- nitrogen (N) of the lysine side chain.

In various embodiments, the self-immolative (SR) moiety includes a disulfide bond (—S—S—), i.e. disulfide bridge with a covalent bond between the two sulfur (S) atoms. Said disulfide bond may provide a biologically relevant precursor to engineer specific intracellular release of the cargo upon exposure to specific conditions. For example, the disulfide bond may be reduced in a reducing environment, such that the disulfide bond is reduced to two thiols (—SH), i.e. dithiols, and trigger the autocatalytic cleavage of the self-immolative (SR) moiety. In various embodiments, the self-immolative (SR) moiety thus comprises a disulfide group that separates upon reduction into two thiols, with one being still attached to the lysine side chain and the other being released. The one thiol remaining on the lysine side chain then autocatalytically cleaves itself off such that the unmodified lysine side chain amino remains.

In various embodiments, the self-immolative (SR) moiety is an organic group with up to 20 carbon atoms. In various embodiments, it comprises the group of the formula —C(═O)—O—(CH₂)ₙ—S—S—R, with the carbonyl C being attached to the epsilon N of the lysine side chain and n being an integer from 1 to 10, preferably 1, 2, 3, 4 or 5, in particular 2 or 3. In such embodiments, R may include, or may be any organic moiety with 1 to 20 carbon atoms, such as, without limitation substituted or unsubstituted alkyl, alkenyl, cycloalk(en)yl, and aryl.

“Alkyl”, as used herein, relates to a linear or branched alkyl group with 1 to 10 carbon atoms, preferably 1 to 6 carbon atoms, such as, without limitation, methyl, ethyl, n-propyl, isopropyl, t-butyl, n-butyl, and 2-butyl. If substituted, the substituent may be selected from the group consisting of —OR¹, —C(═O)R¹, —OC(═O)R¹, —C(═O)OR¹, halogen, such as fluorine, chlorine and bromine, —N₃, with R¹ being selected from unsubstituted or halo-substituted C₁₋₄ alkyl or alkenyl, unsubstituted or halo-substituted C₅₋₆ cycloalk(en)yl, or unsubstituted or halo-substituted C₆₋₁₄ aryl. It can be preferred that the substituent is not a charged group.

“Alkenyl”, as used herein, refers to the alkyl groups that comprise at least one C-C double bond, such as, without limitation, ethenyl (vinyl), 2-propenyl (allyl), and 2-butenyl. If substituted, the substituents are defined as for alkyl above.

“Cycloalk(en)yl”, as used herein, refers to cyclic, non-aromatic alkyl or alkenyl groups, such as without limitation, cyclohexyl. If substituted, the substituents are defined as for alkyl above.

“Aryl”, as used herein, refers to cyclic aromatic groups with 6 to 14 carbon atoms, such as phenyl. If substituted, the substituents are defined as for alkyl above.

In various embodiments, R may include, or may be, —(CH₂)ₙ—O—C(═O)—R′, wherein n is an integer from 1 to 10, for example 1, 2, 3, 4, or 5, and wherein R′ is selected from: C₁₋₄ alkyl, C₆-aryl, preferably phenyl, said alkyl or aryl being unsubstituted or substituted with halogen, such as pentafluorophenyl and trifluoromethyl. In various such embodiments, R may be selected such that the group of formula —C(═O)—O—(CH₂)ₙ—S—S—R is symmetrical, i.e. is —C(═O)—O—(CH₂)ₙ—S—S—(CH₂)ₙ—O—C(═O)—R′, with n being identical on both occurrences.

The self-immolative (SR) moieties disclosed above mask the charge of the lysine side chain and render the residue highly hydrophobic. This in turn changes the phase separation behaviour of the peptides such that they become able again to form coacervates under neutral pH conditions.

The self-immolative moiety (SR) described above may upon reduction release the compound HS-R, such as HS—(CH₂)ₙ—O—C(═O)—R′. Said reduction may occur in the reducing environment in a cell, such as a cell’s cytoplasm. Said release yields the group —C(═O)—O—(CH₂)ₙ—SH that remains bound to the side chain N of the lysine residue (K). The nucleophilic attack of the thiol group on the carbonyl carbon results in cyclisation and autocatalytic cleavage of this group from the lysine side chain, with the reformed amino group becoming, under physiological circumstances, positively charged. Said positive charge causes disassembly of the coacervate. In sum, exposing the coacervate formed from the HBpep peptides with the modified lysine side chain under neutral pH and oxidative conditions, such as outside of a cell, to the reducing environment of a cell’s interior leads to cleavage of the disulfide bond, which in turn leads to autocatalytic cleavage of the rest of the moiety from the lysine side chain and restitution of the side chain amino group, which becomes charged under physiological pH conditions. Said charged lysine residue (K) then destabilizes the peptide coacervate such that it disassembles and any cargo recruited therein is released.

In various embodiments, compositions for delivery of an active agent, such as a pharmaceutical or diagnostic agent, may include a peptide coacervate, said peptide coacervate including one or more (modified) isolated peptides (HBpep-K, HBpep-SR) of the above, and said active agent, wherein the active agent may be recruited in the peptide coacervate.

The term “coacervate”, as used herein, has the meaning as commonly understood in the art and briefly discussed in the background section. Accordingly, coacervates are two-phase liquid compositions, i.e. exhibiting LLPS, comprising or consisting of a concentrated macromolecule-rich (or coacervate) phase and a dilute macromolecule-depleted phase. The two phases of the peptide coacervates are one peptide-rich coacervate phase and one dilute peptide-depleted phase. The peptide-rich coacervate phase is also referred to herein as “peptide coacervate (micro)droplets”.

The term “recruit”, as used herein, in relation to the active agent, means that the active agent is entrapped in the peptide coacervate phase, for example, the peptide coacervate microdroplets formed by the peptides, for instance, the (modified) isolated peptides (HBpep-K, HBpep-SR). The entrapment is such that the active agent is completely surrounded by the (modified) isolated peptides (HBpep-K, HBpep-SR) forming the coacervate phase. In various embodiments, the recruitment of the active agent is an almost instantaneous process occurring over a short time frame, for example, in a few minutes (vs. encapsulation for example, which generally requires a longer period of time). Thus, the active agent is almost immediately incorporated into the peptide coacervate phase, such that it is entrapped by the (modified) isolated peptides (HBpep-K, HBpep-SR).

In various embodiments, the self-immolative (SR) moiety autocatalytically cleaves itself upon exposure to specific conditions, selected from the group consisting or comprising of: pH changes, redox changes, exposure to release agents, such as glutathione (GSH), specifically, cell endogenous GSH which is ubiquitous in cells, and combinations thereof. Depending on the specific condition used, the release mechanism may differ. One type of release agent leads to a basification of the environment of the coacervate phase, with the increase in pH triggering the break of the disulfide bond of the self-immolative (SR) moiety. Other release agents include redox changes by providing a reducing environment, for example, through the exposure to specific reducing agents, such as GSH, i.e. cell endogenous GSH, or exposure to the reductive cytoplasmic milieu. The break or reduction in the disulfide bond of the self-immolative (SR) moiety results in one thiol group being released and the other attached to the lysine side chain. The remaining one thiol group on the lysine side chain then autocatalytically cleaves itself off such that the amino group of the lysine residue (K) is reformed, and the resulting restoration of the charged lysine residue (K) destabilizes the peptide coacervate leading to the subsequent dissolution of the coacervate phase and release of the recruited active agent. As mentioned above, at neutral pH or under oxidative conditions, the isolated peptides (HBpep-K) remain at the single phase, i.e. as monomeric isolated peptides (HBpep-K) in solution, and releases the recruited active agent.

In various embodiments, reducing agents comprise but is not limited to, GSH, ß-mercaptoethanol (BME), dithiothreitol (DTT). Other reducing agents which result in a change in the redox environment may be used, as selected by those skilled in the art. In various embodiments, reducing agent GSH, i.e. cell endogenous GSH, which is abundantly present in cytoplasmic milieu, i.e. cytosol, triggers a thiol-disulfide exchange reaction such that the disulfide bond is reduced to two thiols - one released and the other attached to the peptide. The nucleophilic attack of the remaining thiol group on the carbonyl carbon results in the cyclisation and autocatalytic cleavage of said group from the lysine side chain. Under physiological conditions (i.e. neutral pH such as in the cell’s interior), the restored lysine residue (K) is positively charged and as a result, disassembles to release the recruited active agent directly into cytosol. Thus, the redox-responsive disulfide bonds of the self-immolative (SR) moiety take advantage of extracellular (GSH concentration 2 - 10 µM in body fluids) and intracellular GSH gradients (1 - 10 mM in cytosol) for the delivery of the active agent.

In general, the release of the active agent may for example, be a burst release where essentially the total load of the active agent is released over a short time frame, or may be a sustained release where release occurs over a prolonged duration. Generally, the release occurs within several minutes but may take up to several weeks or days. The release may also be step-wise such that upon exposure to specific conditions, the release starts but stops once said conditions are removed. It may then re-start again once those conditions for release are again met. Such conditions may be tailored to facilitate a step-wise, or need dependent release and are not limited to pH changes, redox changes, and/or exposure to release agents (e.g. reducing agents, such as cell endogenous GSH). In various embodiments, it is preferred that intracellular release may be a burst or sustained release in the presence of reducing agent GSH, i.e. cell endogenous GSH.

In various embodiments, the active agent may, for example, be a pharmaceutical or diagnostic agent, for example, a macromolecular therapeutic agent. Generally, it may be or include, but is not limited to, proteins, (poly)peptides, carbohydrates, nucleic acids, lipids, chemical compounds, nanoparticles. Suitable proteins and polypeptides include antibodies, antibody fragments, antibody variants and antibody-like molecules. “Antibodies”, as used herein, refers to immunoglobins comprising antigen-binding site(s) and includes monoclonal and polyclonal antibodies comprising the various isotypes IgG, IgM, IgD, IgA, IgE. In some embodiments, antibodies may be or include, but is not limited to, recombinant antibodies or recombinant antibody fragments, such as Fab or scFv fragments.

Suitable nanoparticles include those, such as but not limited to, metal nanoparticles, metal oxide nanoparticles and combinations thereof. The nanoparticles may be magnetic nanoparticles. “Nanoparticles”, as used herein, refer to particles that have dimensions, such as the equivalent spherical diameter (ESD), referring to the diameter of a perfect sphere of equivalent volume as the potentially irregular shaped particle, in the nanometer range, typically up to 500 nm, for example up to 250 or up to 100 nm. The nanoparticles may be substantially spherical in shape in a non-limiting embodiment. “(small) Chemical compounds”, as used in this context, relates in particular to molecules, for example, molecules of varying molecular weights, for example, organic compounds with a molecular weight ranging from 5 kDa to 600 kDa, or ranging from 10 kDa to 500 kDa. This group of compounds includes ribosome inactivating protein, saporin. A pharmaceutical agent from the group of (poly)peptides includes peptide hormones. Further, pharmaceutical agents from the group of peptides includes the second mitochondria-derived activator of caspases peptide (Smac) and proapoptotic domain peptide (PAD). “Polypeptides”, as used herein, relates to polymers of amino acids connected by peptide bonds. Molecules that comprise multiple polypeptide chains, typically connected by non-covalent interactions or cystine bridges, are referred to as “proteins” herein. Polypeptides typically comprise more than 100, for example, 200, or 500 amino acids or more, and includes polypeptides of varying molecular weights and isoelectric points. The term polypeptide/protein as used herein also comprises antibodies, antibody fragments and antibody-like proteins or polypeptides. A pharmaceutical agent from the group of polypeptides/proteins include the antimicrobial and antiviral lysozyme enzyme. Diagnostic agents from the group of polypeptides/proteins include bovine serum albumin (BSA), phycoerythrin (R-PE), enhanced green fluorescence protein (EGFP), β-galactosidase (β-Gal), either alone or in combinations thereof.

In various other embodiments, the pharmaceutical or diagnostic agent may include or be, but is not limited to, RNA oligonucleotides or variants thereof, such as, plasmid DNAs, small interfering RNAs, microRNAs, messenger RNAs, long non-coding RNAs, and other RNA oligonucleotides such as those used in CRISPR / Cas9 or other genome-editing systems. “mRNA”, as used in this context, relates to single-stranded RNA molecules corresponding to the genetic sequence of a gene, and is read by a ribosome in the process of protein synthesis, i.e. during translation. In some embodiments, the pharmaceutical or diagnostic agent is luciferase-encoding mRNA, EGFP-encoding mRNA, either alone or in combinations thereof.

In various embodiments, the pharmaceutical or diagnostic agent comprises or is, but is not limited to, anti-cancer agents, including macromolecular anti-cancer agents, such as proteins and/or peptides, including antibodies, as well as fragments and variants thereof. In some embodiments, the pharmaceutical agent comprises or is, but is not limited to, agent(s) such as saporin, and small peptides such as the anti-cancer stapled peptides, Smac and PAD peptides, either alone or in combination. In some embodiments, the pharmaceutical or diagnostic agent, saporin, Smac peptide and PAD peptide, is recruited in the peptide coacervate either alone or in combinations thereof. The active agent is released from the peptide coacervate upon exposure to the specific conditions discussed above. In some embodiments, release of the active agent is facilitated by the exposure of the peptide coacervate to redox changes, in particular the reducing environment in the cytosol of the cell and/or GSH, i.e. cell endogenous GSH, as a reducing agent.

In various embodiments, the composition comprises a pharmaceutical or diagnostic formulation for administration to a subject. Such formulations may additionally comprise all the known and accepted additional components for such applications. These include auxiliaries, carriers and excipients that are pharmaceutically or diagnostically acceptable, for example various solvents, preservatives, dyes, stabilizers and the like. Such formulations may additionally comprise further active agents that are not recruited in the peptide coacervate phase. In various embodiments, such compositions are liquid compositions, including gels and pastes. “Liquid”, as used herein, particularly refers to compositions that are liquid under standard conditions, i.e. 20° C. and 1013 mbar. In various embodiments, such liquid compositions are pourable. The compositions may be in single dose or multi dose form. Suitable forms and packaging options are well known to those skilled in the art.

In various embodiments, the composition can be adapted for administration to a mammalian subject, for example, a human being.

In various embodiments, the peptide coacervates comprising the one or more (modified) isolated peptides (HBpep-K, HBpep-SR) is in the form of colloids recruiting the active agent. In various embodiments, the colloidal phase has the form of (micro)droplets having a substantially spherical shape with a diameter ranging from about 0.5 µm to about 5 µm, or 0.8 µm to 2 µm, for example about 1 µm. The diameter of the substantially spherical shape may be the ESD, referring to the diameter of a perfect sphere of equivalent volume as the potentially irregularly shaped (micro)droplet. For example, the (micro)droplet may have an ellipsoid shape, and the equivalent spherical diameter would then be the diameter of a perfect sphere of exactly the same volume. Each of the (micro)droplets are made up of the peptide coacervates and, in various embodiments, is homogeneous in that it has no distinct core-shell morphology, but rather is a colloidal particle with no peptide gradient over its radius. In alternative or additional embodiments, the coacervate phase may take the form of a condensed hydrogel.

As indicated above, the isolated peptides (HBpep-K) comprising the single lysine residue (K) form coacervates at an increased pH of 9.0, which is not suitable for intracellular delivery of the active agent since cytoplasmic milieu is at neutral pH (i.e. pH of about 7.0). At the pH of cytoplasmic milieu, the isolated peptides (HBpep-K) remain as monomeric peptides in solution.

The inventors’ surprisingly found that the modified isolated peptides (HBpep-SR) comprising the self-immolative (SR) moiety conjugated to the amino group of the lysine residue (K), forms coacervates readily, in particular, under neutral conditions at pH of more than 5.0. In various embodiments, the pH of the modified isolated peptides (HBpep-SR) recruiting the active agent ranges from about 5.0 to 8.0, for example, at pH of about 6.0, or at pH of about 6.5. The conjugation of the self-immolative (SR) moiety to the amino group of the inserted lysine residue (K) was able to neutralize the extra positive charge and shift the isoelectric point of the isolated peptide (HBpep-K), thus increasing the hydrophilicity of the unmodified peptides (HBpep), which in turn affects the phase separation behaviour of the modified isolated peptides (HBpep-SR). In other words, the modified isolated peptides (HBpep-SR) were able to recruit the active agent during the self-coacervation process under neutral pH to form peptide coacervates (or colloids). These pH values ensure that the coacervate (or colloidal) phase remains stable. Stable solutions of the modified isolated peptides (HBpep-SR) without any distinct phase separation may be formed under acidic conditions, for example at pH 4.0 or less. In various embodiments, the modified isolated peptides (HBpep-SR) may be prepared as stock solutions in slightly acidic conditions, such as 1 to 100 mN, for example, in about 10 mM acetic acid solution or other suitable weak acids.

Methods of manufacture of the above composition is also disclosed. Methods for the recruitment of an active agent in a peptide coacervate comprise: (1) providing an aqueous solution of coacervate-forming peptides, said coacervate-forming peptides comprising one or more modified isolated peptides (HBpep-SR) of the invention, (2) combining the aqueous solution of the coacervate-forming peptides with an aqueous solution of an active agent, and (3) inducing coacervate formation.

The term “aqueous solution”, as used herein, means that the dilute phase is mainly water, i.e. comprises at least 50 vol.% water. In various embodiments, the composition may use water as the only solvent, i.e. no additional organic solvents, such as alcohols, are present. In other embodiments, the composition is an aqueous composition that additionally contains one or more solvents other than water, with water however being the major constituent, i.e. being present in an amount of at least 50, at least 60, at least 70, at least 80, at least 90, at least 95 or 99 vol. %.

As mentioned above, the modified isolated peptides (HBpep-SR) may be dissolved in a weak acid, for examples aqueous acetic acid, of a concentration of 1 to 100 mM, such as 10 mM. Other weak acids may be equally suitable as long as the coacervate-forming modified isolated peptides (HBpep-SR) remain stable in solution, and such acids may be routinely selected by those skilled in the art. In these embodiments, the pH of the aqueous solution of the coacervate-forming modified isolated peptides (HBpep-SR) may be below pH 5.0, for example, below 4.5 or below 4.0. The pH is however, in various embodiments, higher than pH 0, for example pH 1.0 or higher, such as pH 2.0 or higher.

In various embodiments, for forming the peptide coacervate and at the same time recruiting the active agent, the solution of the coacervate-forming modified isolated peptides (HBpep-SR) is combined with the active agent and coacervate formation is induced. In various embodiments, the induction of coacervate formation is induced by increasing the pH of resulting solution containing both the coacervate-forming modified isolated peptides (HBpep-SR) and the active agent, as well as optionally, the additional components and/or auxiliaries. The pH is increased to values of 5.0 or more, for example, 5.5 or more, or 6.0 or more. It was found that the optimal pH to effect coacervate microdroplets is at pH of about 6.5 or more, and in various embodiments, not higher than pH 8.0. To maintain such pH to induce coacervate formation, the active agent is dissolved or diluted in a suitable buffering agent, for example, a buffering agent with a pH between 6.0 to 7.5, for example, phosphate buffers with a pH of 6.5, such that the combined aqueous solution of the coacervate-forming modified isolated peptides (HBpep-SR) and the active agent retains a pH of about 6.0, or about pH 6.5.

In various embodiments, a volume ratio of the aqueous solution of the coacervate-forming peptides to the aqueous solution of the active agent is higher than 1 : 5, but in various embodiments, not higher than 1 : 20, for example, between 1: 8 to 1 : 10. In preferred embodiments, the volume ratio of the aqueous solution of the coacervate-forming peptides to the aqueous solution of the active agent is between 1 : 8 to 1 : 10, for example, at about 1 : 9, or at about 1 : 9.5.

After the coacervate formation, the composition is an aqueous liquid two phase formulation, i.e. a composition comprising (1) a coacervate colloidal phase comprising the modified isolated peptides (HBpep-SR) and the active agent; and (2) a dilute aqueous phase.

Methods of delivery of the active agent, such as pharmaceutical or diagnostic agents, is further disclosed. Methods for the delivery of an active agent comprise: (1) providing a composition comprising a peptide coacervate, the peptide coacervate comprising the modified isolated peptides (HBpep-SR), and an active agent, and (2) exposing the peptide coacervate to conditions that trigger the release of the active agent from the peptide coacervate.

In various embodiments, the provided compositions comprising the peptide coacervate is exposed or subjected to conditions which facilitate the release of the active agent from the coacervate phase. Said release is facilitated by dissolution of the isolated peptides of the coacervate phase, for example, through the autocatalytic cleavage of the self-immolative (SR) moiety from the amino group of the lysine residue (HBpep-K) by suitable means to restore the positively charged lysine residue (K) and the resulting dissolution of the coacervate, i.e. colloid phase. Some of the release mechanisms have been described above, namely, pH changes, redox changes, and/or the exposure to release agents, e.g. reducing agents, such as GSH, i.e. cell endogenous GSH. Additional release mechanisms may be envisioned and may include denaturing agents that disrupt the disulfide bond of the self-immolative (SR) moiety, resulting in the dissolution of the formed coacervate, i.e. colloid phase.

Methods for treating or diagnosing a condition or disease in a subject in need thereof is also disclosed, wherein the compositions described above are used in the treatment and/or diagnosis. Such methods of treatment also include methods where a disease, condition or disorder is managed, for example, in that the symptoms or effects may be alleviated. In various embodiments, the treatment methods include anti-cancer therapies, wherein compounds such as saporin, and peptides such as Smac and PAD peptides, delivered alone or in combination thereof, exhibit cytotoxicity against cancer cells. It is further envisioned that the treatment method may include vaccines for the prevention of a specific diseases.

In the above method, the composition described herein including the peptide coacervate of modified isolated peptides (HBpep-SR) and a pharmaceutical or diagnostic agent recruited in the peptide coacervate, is administered to said subject. Methods of administration may include any suitable administration route including oral administration or parenteral administration, for example intravenous, intramuscular, subcutaneous, epidural, intracerebral, intracerebroventricular, nasal, intraarterial, atraarticular, intracardiac, intradermal, intralesional, intraocular, intraosseous, intravitreal, intraperitoneal, intrathecal, intravaginal, transdermal, transmucosal, sublingual, buccal, and perivascular. In various embodiments, the administration may be systemic or localized, e.g. topically.

In the above method, said pharmaceutical or diagnostic agent is released from the peptide coacervate by exposing the peptide coacervate to conditions that trigger the release of the pharmaceutical or diagnostic agent. In various embodiments, the exposure occurs automatically due to conditions in the body of the subject, such as through metabolic action, which triggers the release of the recruited pharmaceutical or diagnostic agent.

In various embodiments, the subject may be a mammal, for example a human being. Further, the conditions that trigger the release of said agent are generally selected from the above conditions. In particular, upon administration of the composition intracellularly, dissolution of the coacervate phase is facilitated by exposure to naturally occurring reducing agents found in the cell, such as the reducing agent GSH, i.e. cell endogenous GSH, which is abundant in cytosol. GSH reduces the disulfide bond of the self-immolative (SR) moiety into two thiols groups - one attached to the lysine side chain and the other released. The thiol group attached to the lysine side chain then autocatalytically cleaves itself off such that the unmodified charged lysine side chain is restored, resulting in the dissolution of the coacervate phase and release of the recruited pharmaceutical or diagnostic agent.

In a non-limiting embodiment of these methods for the treatment of a disease, the subject is a human afflicted by cancer, the pharmaceutical agent is an anti-cancer therapeutic agent, and release is facilitated by the exposure of the composition to GSH, i.e. cell endogenous GSH. In such embodiments, the composition remains stable in the extracellular environment, i.e. neutral pH or oxidative conditions, for example, in the body fluids of the subject where GSH concentration is low (2 - 10 µM). The peptide coacervates then cross the cell membrane via an endocytosis-independent pathway to directly enter the cytosol, and disassembly of the peptide coacervates is triggered by the reducing environment in the cell’s interior, facilitated by amongst others, intracellular GSH, resulting in the release of the recruited therapeutic agent.

In non-limiting embodiments, the cancer may be liver cancer, colon cancer, lung cancer, prostate cancer, breast cancer, and the like.

It is understood that release of the recruited pharmaceutical or diagnostic agent may also be facilitated by exposure to condition which disrupts the disulfide bond resulting in the autocatalytic cleavage of the self-immolative (SR) moiety, restoration of the charged lysine side chain, and resulting dissolution of the peptide coacervates.

Advantageously, the redox-responsive peptide coacervates presents a novel and safe delivery platform for both the intracellular delivery and direct cytosolic release of a large palette of biomacromolecular therapeutics. Critically, the recruitment process of a therapeutic agent is carried out under aqueous environments, thereby preventing the loss of bioactivity of said therapeutic agent and enhancing safety. The redox-responsive peptide coacervates remain stable at neutral conditions, i.e. neutral pH, enabling intracellular delivery of therapeutic agents which take advantage of extracellular and intracellular GSH gradients. The versatility of cargo recruitment and release makes this intracellular delivery platform a promising candidate for the treatment of cancer, metabolic, and/or infectious diseases.

Additional applications of the compositions and methods will be identifiable by the person skilled in the art. The compositions and methods herein disclosed are further illustrated in the following examples, which are provided by way of illustration and are not intended to be limiting the scope of the present disclosure.

### EXAMPLES

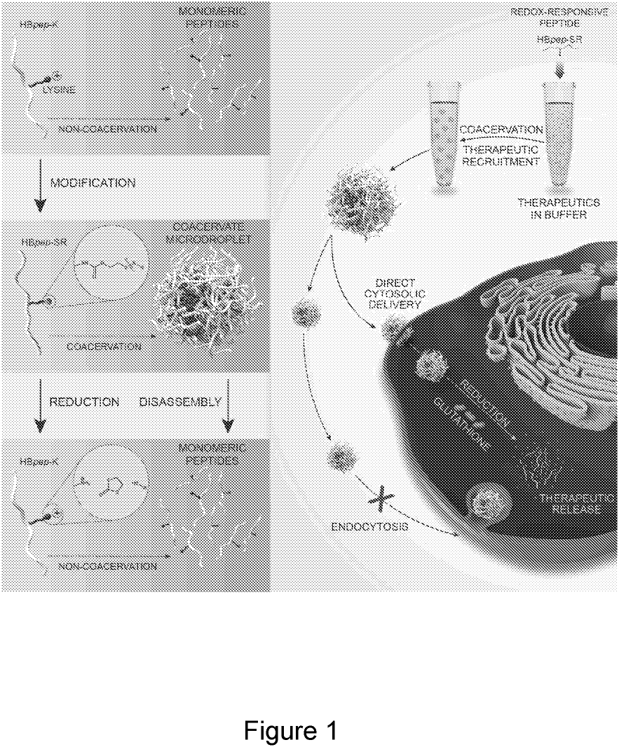

In the design of the examples below, HBpep was modified to create redox-response peptide coacervates (HBpep-SR) with direct cytosolic entry that bypasses endocytosis. FIG. 1 shows a schematic illustration of the intracellular delivery system based on HBpep-SR. Briefly, HBpep is first modified by the insertion of a single lysine (K) residue (HBpep-K). HBpep -K (top left) remains in solution at neutral pH but can phase separate and form coacervates after conjugation of the sole lysine residue (K) with a self-immolative (SR) moiety (HBpep-SR, middle left). In a reducing environment such as the GSH-rich cytosol, HBpep-SR is reduced, followed by auto-catalytic cleavage of the SR moiety, resulting in HBpep-K again and in the disassembly of the peptide coacervates (left bottom). During coacervation of HBpep-SR near neutral pH (top right), macromolecular therapeutics are readily recruited within the coacervates. Upon incubation with cells, the therapeutics-loaded coacervates cross the cell membrane to migrate directly in the cytosol (right bottom), whereupon they are reduced by GSH resulting in the disassembly and release of the therapeutic agent.

In the Examples below, the isolated peptide (HBpep-K) sequence comprising the amino acid sequence GHGVY GHGVY GHGPY K GHGPY GHGLY W (SEQ ID NO: 10) with insertion of a single lysine residue (K) at position 16 from the N-terminal of HBpep was used as the representative isolated peptide sequence in the peptide coacervate composition. NHS—SS—Ac and NHS—SS—Ph, synthesized from acetic acid (Ac) and benzoic acid (Ph), respectively, were used as representative self-immolative (SR) moieties.

### Experimental Details

### Materials

Resins and Fmoc protected amino acids used in solid phase peptide synthesis were purchased from GL Biochem, China. N-Hydroxysuccinimide (NHS), tetrahydrofuran, triphosgene, sodium azide and benzoic acid were purchased from Tokyo Chemical Industry (TCI), Japan. N,N′-Diisopropylcarbodiimide, acetic acid, 2-hydroxyethyl disulfide, N,N-diisopropylethylamine, piperidine, trifluoroacetic acid, triisopropylsilane, 2,4,6-trinitrobenzenesulfonic acid, 1,4- dithiothreitol (DTT), glutathione (GSH), bovine serum albumin (BSA), lysozyme, insulin, saporin, β-galactosidase (β-Gal), R-phycoerythrin (R-PE), methylthiazolyldiphenyl-tetrazolium bromide, Hoechst 33342, methyl-β-cyclodextrin, chlorpromazine hydrochloride, amiloride chloride were obtained from Sigma-Aldrich, USA. Dichloromethane, N,N-dimethylformamide, LysoTracker Red DND-99, Opti-MEM, Ni-NTA His bind resin and 5-bromo-4-chloro-3-indolyl β-D-galactopyranoside were purchased from Thermo Fisher Scientific, USA. Organic solvents including ethyl acetate, hexane and diethyl ether were purchased from Aik Moh Paints & Chemicals Pte Ltd, Singapore. Dulbecco’s modified Eagle medium, fetal bovine serum, phosphate buffered saline and Antibiotic-Antimycotic (100X) liquid were purchased from Gibco, USA. Nano-Glo® Dual-Luciferase® kit used for luciferase detection was purchased from Promega, USA. Enhanced green fluorescent protein (EGFP) was expressed by E. Coli BL21 strain and purified with Ni-NTA His bind resin. Luciferase-encoding mRNA encoded and EGFP-encoding mRNA used for mRNA transfection experiments were obtained from Trilink.

### Peptide Synthesis and Purification

The peptides used in this study were synthesized by the classical Merrifield solid phase peptide synthesis (SPPS) technique (Merrifield, R.B., J. Am. Chem. Soc., 1963, 85, 2149). Wang resin (1.0 g, 0.56 mmol) was first swollen in 15 mL of dichloromethane (DCM) for 0.5 hours with nitrogen flow bubbling. Then, the DCM was drained with increased pressure, and the resin was washed three times with DMF.

For N-terminal protected amino acid (Fmoc-AA-OH) coupling, Fmoc-AA-OH (2 equiv, 1.12 mmol) was dissolved in 5 mL of N,N-dimethylformamide (DMF), then 5 mL of DMF with 1-[Bis(dimethylamino)methylene]-1H-1,2,3-triazolo[4,5-b]pyridinium 3-oxide hexafluorophosphate (HATU, 1.9 equiv, 1.064 mmol) and DIPEA (5 equiv, 2.80 mmol) was added into the prior solution. The mixture was reacted for 2 min at room temperature before being added onto the resin for 1 hour of coupling reaction with nitrogen flow bubbling. The resin was washed with DCM and then DMF three times each after the coupling reaction. The coupling efficiency was evaluated by using 2,4,6-trinitrobenzenesulfonic acid (TNBS).

For deprotection of N-terminal amine, 15 mL of 20% piperidine in DMF (volume ratio) was added onto the resin. The deprotection continued for 0.5 hour at room temperature with nitrogen flow bubbling. After that, the resin was washed with DCM then DMF three times and the deprotection efficiency was also evaluated using 2,4,6-trinitrobenzenesulfonic acid (TNBS).

After all amino acids in the peptide sequence were coupled onto the resin by performing coupling/deprotection cycles from the C- to the N- termini direction, the peptides were cleaved from the resins by using a cocktail containing 95% of trifluoroacetic acid (TFA), 2.5% of H₂O and 2.5% of triisopropylsilane (TIPS). After 2 hours of cleavage, the reaction mixtures were filtered. The supernatants were concentrated by using nitrogen flow and precipitated into 50 mL of cold diethyl ether. After centrifugation, the pellets were dried under vacuum and re-dissolved by using 90% of 10 mM acetic acid and 10% acetonitrile for purification by High Performance Liquid Chromatography (HPLC, 1260 Infinity, Agilent Technologies, USA) equipped with a C8 column (Zorbax 300SB-C8, Agilent Technologies, USA). The purified peptides were isolated by lyophilization (FreeZone 4.5 Plus, Labconco, USA) from HPLC elutes.

### Self-immolative Moiety Synthesis

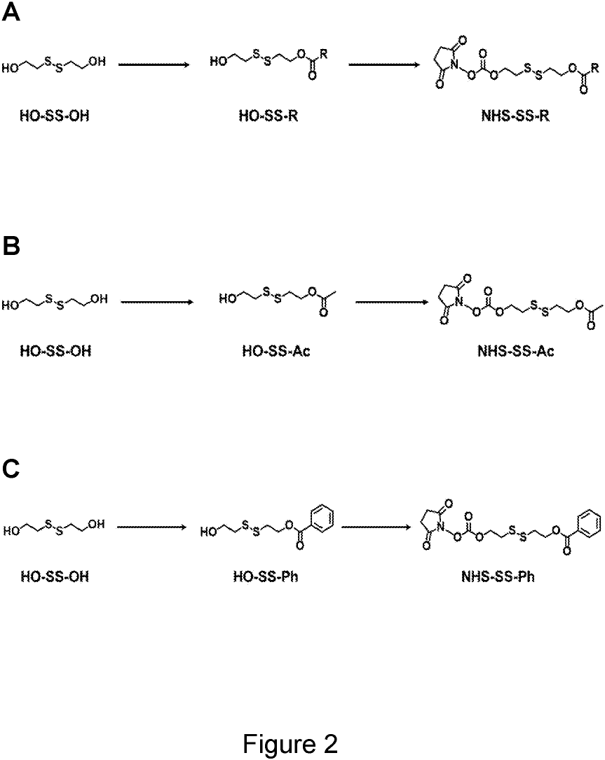

The self-immolative (SR) moieties conjugated to HBpep-K peptides were designed based on the literature (Tang, L. et al., Nat. Biotech., 2018, 36, 707), and the synthesis routes of the amine-reactive species are shown in FIG. 2. First, for the synthesis of the side blocked intermediate product (FIG. 2A), HO—SS—R, 2-hydroxyethyl disulfide (1 equiv, 10 mmol) was dissolved in 15 mL tetrahydrofuran (THF), and another 15 mL THF containing a carboxylic acid reactant including acetic acid and benzoic acid (0.9 equiv, 9 mmol) was added. Then, under an ice bath, 15 mmol of N,N′-Diisopropylcarbodiimide (DIC) was slowly added into the reaction mixture. The reaction was kept at 0° C. for another 0.5 hours and then increased to room temperature. After the overnight reaction, the mixture as filtered, and the supernatant was evaporated under reduced pressure. The raw products were then purified using silica gel chromatography with ethyl acetate/hexane (¼) as elute. The purified products were by rotary evaporation (R-215 Rotavapor, BUCHI, Switzerland).

Then, the intermediate products HO—SS—R and N-hydroxysuccinimide (NHS) were coupled by using triphosgene (FIG. 2A). Specifically, HO—SS—R (1 equiv, 5 mmol), and 4-dimethylaminopyridine (DMAP, 0.1 equiv, 0.5 mmol) was dissolved in 10 mL of THF. Then, triphosgene (0.37 equiv, 1.85 mmol) in 10 mL THF was added into the prior solution dropwise under an ice bath. After another 0.5 hours on the ice bath, the reactions were continued at 40° C. for 4 hours, followed by evaporation under reduced pressure to remove excess phosgene. NHS (1.5 equiv, 7.5 mmol) in 20 mL THF, and N,N-Diisopropylethylamine (DIEPA, 1.5 equiv, 7.5 mmol) was then pipetted in the prior mixtures. The reactions were kept at 40° C. for 24 hours before evaporation. The raw products were purified using silica gel chromatography with ethyl acetate/hexane (⅓) as elute. The purified products were isolated by rotary evaporation. The amine-reactive products NHS—SS—Ac and NHS—SS—Ph were synthesized from acetic acid (labelled as “SA” below, FIG. 2B) and benzoic acid (labelled as “SP” below, FIG. 2C).

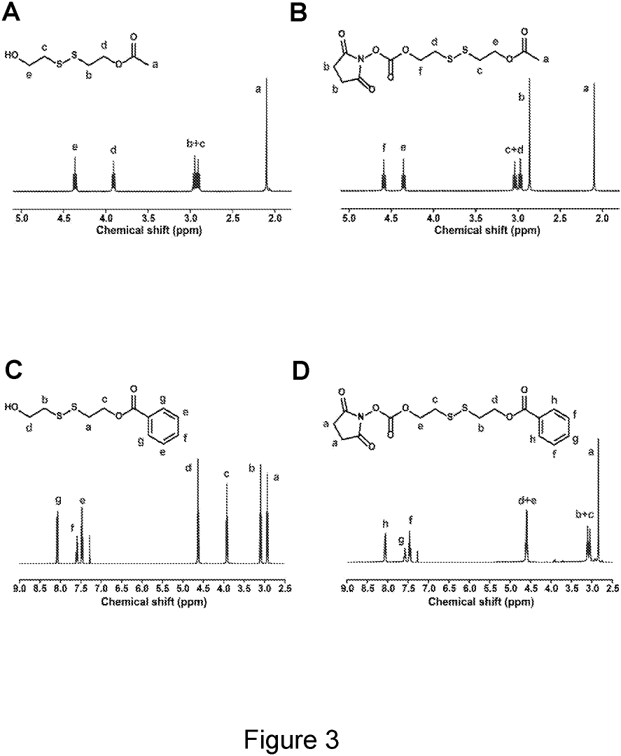

The chemical structures of the HO—SS—R and NHS—SS—R were verified by ¹H nuclear magnetic resonance (NMR) as shown in FIG. 3. Synthesized products were dissolved in chloroform (CDCl₃) solvent, and the NMR spectra were collected on a Bruker Advance 400 spectrometer (USA). The chemical shifts and areas under the peaks of the ¹H NMR spectra of HO—SS—Ac (FIG. 3A) and NHS—SS—Ac (FIG. 3B), and HO—SS—Ph (FIG. 3C) and NHS—SS—Ph (FIG. 3D) suggested the successful synthesis of the self-immolative (SR) moieties.

### Peptide Modification

The redox responsive peptides were synthesized by reacting the epsilon (ε)-amine of the single lysine residue (K) of the N-terminal protected peptide (Fmoc-HBpep-K, Fmoc-GHGVY GHGVY GHGPY K GHGPY GHGLY W (SEQ ID NO: 10)) with the amine-reactive species NHS—SS—R, followed by deprotection. First, the Fmoc-HBpep-K peptide (1 equiv, 15 µmol) was dissolved in 5 mL of DMF containing DIPEA (15 equiv, 225 µmol). After 30 minutes of deprotonation, NHS—SS—R (1.5 equiv, 22.5 µmol) in 0.5 mL of DMF was added into the solution. The mixture solutions were allowed to react at room temperature for 24 hours before precipitation by adding 50 mL of cold diethyl ether. The raw products were collected from the pellets by centrifugation, and dried under reduced pressure. The purification of modified peptides was conducted on an HPLC system equipped with a C8 column. The purified Fmoc protected peptides were isolated by lyophilization from the HPLC fractions.

Then, the purified Fmoc protected peptides were dissolved in 5 mL of DMF containing 20% piperidine. The mixture was stirred at room temperature for 2 hours of N-terminal deprotection. The raw products were collected from the precipitates after adding 50 mL of cold diethyl ether into the reaction mixtures and purified by HPLC. The final products were isolated by lyophilization as white solids. Two modified peptides were synthesized, namely HBpep-SA from NHS—SS—Ac and HBpep-SP from NHS—SS—Ph. The modified peptides HBpep-SA and HBpep-SP were dissolved in 10 mM acetic acid solution at 10 mg/mL as stock solution.

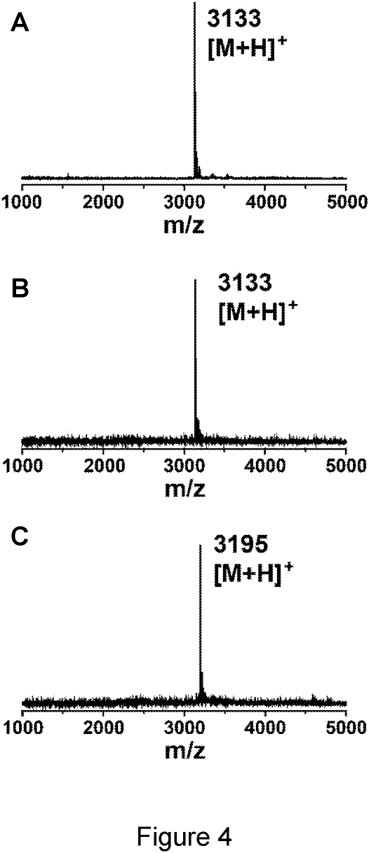

The molecular weights (MW) of Fmoc-HBpep-K and modified peptides were verified by matrix assisted laser desorption ionization-time of flight (MALDI-TOF) mass spectrometry, using α-cyano-4-hydroxycinnamic acid (CHCA) as the matrix (FIG. 4). The MALDI-TOF spectra were collected on an AXIMA Performance spectrometer (Shimadzu Corporation, Japan). The MWs of the HBpep-SA (FIG. 4B) and HBpep-SP (FIG. 4C) conjugated peptides were consistent with the expected MWs of the peptides, when compared to the MW of the Fmoc-HBpep-K (FIG. 4A).

### Coacervation of Modified Peptide

The phase separation behavior of HBpep-K and HBpep-SR peptides at various pH was monitored turbidity measurements using a UV-Vis spectrometer (UV-2501PC, Shimadzu, Japan). The absorbance at 600 nm (A600) was used to calculate the relative turbidity (Lim, Z.W. et al., Bioconjugate Chem., 2018, 29, 2176) as:

$$100 - 100 ∗ \left({10}^{- A 600}\right) .$$

### Therapeutic Recruitment

The recruitment of the macromolecules within the peptide coacervates was conducted during the coacervation process at the optimal pH of 6.5. The therapeutics were dissolved or diluted in 10 mM phosphate buffers (pH = 6.5, ionic strength = 100 mM) to achieve the target concentrations. Then, the peptides stock solutions were mixed with the therapeutics containing the buffer at a 1 : 9.0 volume ratio to induce coacervation and recruitment of the therapeutics. The recruitment efficiency of proteins was calculated by comparing the supernatant fluorescence in the buffer solution before and after coacervation using microplate reader (Infinite M200 Pro, Tecan, Switzerland). The fluorescence of EGFP (or FITC) and R-PE were detected using 488 nm / 519 nm and 532 nm / 584 nm for the excitation / emission wavelengths, respectively. In the latter case, the measurement was done after the centrifugation step used to recover the coacervates.

### Characterization of Redox-Responsive Peptide Coacervates

Optical and fluorescence microcopy images of HBpep-SP coacervates and fluorescence image of macromolecules-loaded HBpep-SP coacervates were taken using an invert fluorescence microscope (AxioObserver.Z1, Zeiss, Germany). Dynamic light scattering (DLS, ZetaPALS, Brookhaven, USA) system was employed to measure the size of pristine HBpep-SR coacervates and macromolecules-loaded HBpep-SR coacervates. The fresh prepared pristine or macromolecules-loaded coacervates (with or without 0.1 mg/mL of macromolecules, 1 mg/mL of modified peptides) was diluted into PBS with a volume ratio of 1 : 9.0 before the DLS test.

### In Vitro Insulin Release in the Presence of DTT

The redox-responsive property of the HBpep-SA and HBpep-SP was first tested in an in vitro release study using FITC-labeled insulin, which was released from dialysis tubes in the presence of DTT. Specifically, 5 µL of HBpep-SR stock solutions were gently mixed with 45 µL of buffers containing 0.1 mg/mL FITC-insulin. The mixtures were then transferred into dialysis tubes with another 150 µL of PBS. The dialysis tubes were placed into a 15 mL centrifuge tube against 1 mL of PBS in the presence or absence of 10 mM DTT. The solutions outside of dialysis tubes were collected and replaced with fresh DTT / PBS or PBS at various time points. The percentage of released FITC-insulin was measured with a microplate reader and calculated based on a calibration curve.

### In Vitro Macromolecule Release in the Presence of GSH

The redox-responsivity of HBpep-SA and HBpep-SP was next evaluated by measuring the decrease in concentration in the presence of GSH. The fresh prepared HBpep-SA or HBpep-SP coacervates (50 µL, 1 mg/mL of peptide) were diluted in 450 µL of PBS containing 1 mM of GSH. The mixtures were incubated at 37° C. before adding 25 µL of acetic acid to dissolve all the unreacted peptides, and their concentration was measured by HPLC.

### Delivery of Proteins and Peptides

For protein delivery into cells, 10⁵ of cells were suspended in 1 mL of Dulbecco’s modified Eagle medium (DMEM) supplemented with 10% of fetal bovine serum, 100 units/mL of penicillin and 100 µg/mL of streptomycin, and then transferred into 35 cm² culture dishes. After 24 hours of incubation at 37° C. with 5% of CO₂, the media was replaced with 900 µL of Opti-MEM. Then, 100 µL of freshly prepared protein-loaded HBpep-SA or HBpep-SP coacervate suspensions (0.1 mg/mL of cargos, 1 mg/mL of modified peptides) were added into the media. After 4 hours of incubation, the media was removed and the cells were washed with PBS twice before adding 1 mL of fresh media (DMEM, 10% FBS, antibiotics). The cells were incubated for another 20 hours and then washed twice at pH 5.0 in phosphate buffer to remove any coacervates that had not entered the cells, before being imaged under the fluorescence microscope (AxioObserver.Z1, Zeiss, Germany).

### Delivery and Transfection of mRNA Proteins and Peptides

Two reporter genes including luciferase and EGFP, were used to evaluate the mRNA transfection efficiency of the HBpep-SR coacervates. Before transfection, HepG2 or HEK293 cells were incubated in a 96-wells plate with a density of 10⁴ cells per well for 24 hours. Then, the media were replaced with 90 µL of Opti-MEM, followed by the addition of 10 µL of freshly prepared mRNA-loaded coacervate suspensions (1 or 2 mg/mL of modified peptides). The final concentration of luciferase-encoding mRNA used in transfection was 3.3 µg/mL. After 4 hours of incubation, the media were removed and the cells were washed by PBS twice before adding 100 µL of media (DMEM, 10% FBS, antibiotics). Then transfection was continued for another 20 hours before testing the luminescence using the Nano-Glo® Dual-Luciferase® kit and a microplate reader. For EGFP-encoding mRNA labeled with Cy5 transfection, the cultures were conducted in 35 cm² dish in which 100 µL of mRNA loaded HBpep-SP coacervates (1 mg/mL of HBpep-SP) was added to achieve the final mRNA concentration of 1 µg/mL The transfection was conducted for 4 hours of uptake and 20 hours of expression before imaging the cells under a fluorescence microscope and testing the transfection efficiency by FACS (LSR Fortessa X20, BD Biosciences, USA).

### Cytotoxic Study

The cytotoxicity of the therapeutics-loaded or pristine peptide coacervates was evaluated by using the methylthiazolyldiphenyl-tetrazolium bromide (MTT) assay. Following literature protocols (Chang, H. et al., Nano Letters, 2017, 17, 1678; Sun, Y. et al. Biomat., 2017, 117, 77), 10⁴ of HepG2 or HEK293 cells in 100 µL of media (DMEM, 10% FBS, antibiotics) were transferred into 96-wells plates and incubated for 24 hours. Then, the media were replaced with 100 µL of Opti-MEM containing therapeutics-loaded coacervates (various concentration of therapeutics, 1 mg/mL HBpep-SP) or various concentrations of pristine coacervate suspensions. After 4 hours of uptake, the media were removed and the cells were washed by PBS twice before adding 100 µL of media (DMEM, 10% FBS, antibiotics). The cells were incubated for another 20 hours before 10 µL of 5 mg/mL MTT dissolved in PBS was added. The media were removed after 4 hours of incubation with MTT, and the cells were washed by PBS twice. After that, 100 µL of DMSO per well was added for absorbance measurements at 570 nm using a microplate reader (Infinite M200 Pro, Tecan, Switzerland). The relative cell viability was calculated as:

$$\frac{{\text{A}}_{\text{t}} - {\text{A}}_{\text{b}}}{{\text{A}}_{\text{c}} - {\text{A}}_{\text{b}}} ∗ 100 \% ,$$

where Aₜ, A_{b}, and A_{c} represent the absorbance of tested cells, control cells and no cell, respectively.

### Internalization Mechanism Study

The LysoTracker staining was conducted by following the manual from the manufacturer. Similar to protein delivery, 10⁵ of HepG2 cells were incubated in 35 cm² dish with DMEM for 24 hours. Then the media were replaced with 900 µL of Opti-MEM and 100 µL of EGFP-loaded HBpep-SP coacervates (0.1 mg/mL of EGFP, 1 mg/mL of HBpep-SP). The cells were cultured for another 2 hours before being washed twice with a pH 5.0 phosphate buffer to remove any coacervates that had not entered the cells. After that, 1 mL of Opti-MEM containing 50 nM of LysoTracker was added for 30 minutes of staining at cell culture condition. The treated HepG2 cells were washed by PBS twice and fixed with 4% formaldehyde solution. Before being imaged by confocal microscopy (LSM 780, Zeiss, Germany), the cells were treated with 1 µg/mL of Hoechst 33342 for 10 minutes to stain the nucleus.

Based on the literature (Mout, R. et al., ACS Nano, 2017, 11, 2452; Xu, C. et al., Int. J. of Pharma., 2015, 493, 172; Lin, Q. et al., Pharma. Res., 2014, 31, 1438), various inhibitors were used to study the pathway of the coacervates internalization. HepG2 cells were treated with chlorpromazine (CPM, 30 µM), amiloride chloride (AM, 20 µM), sodium azide (NaN₃, 100 mM) or methyl-β-cyclodextrin (MβCD, 2.5 mM) separately for 1 hour. Then the 100 µL of EGFP loaded HBpep-SP coacervates (0.1 mg/mL of EGFP, 1 mg/mL of HBpep-SP) was added. After another 4 hours of incubation, the cells were washed twice with a pH 5.0 phosphate buffer followed by PBS thrice. Then the treated cells were imaged by fluorescence microscopy or dissociated by trypsin for FACS. For the 4° C. treated group, the HepG2 cells were preincubated for 1 hour and kept at low temperature during the 4 hours of uptake process. Two control groups including totally untreated cells (control) and cells treated by EGFP-loaded coacervates without any inhibitors (blank) were also conducted.

### Statistical Analysis

All experiments were repeated three times. The data are presented as means ± standard deviation (SD). Statistical significance (p < 0.01) was evaluated by using two-sided Student’s t-test when only two groups were compared.

### Example 1: Characterization of Redox-responsive Peptide Coacervates

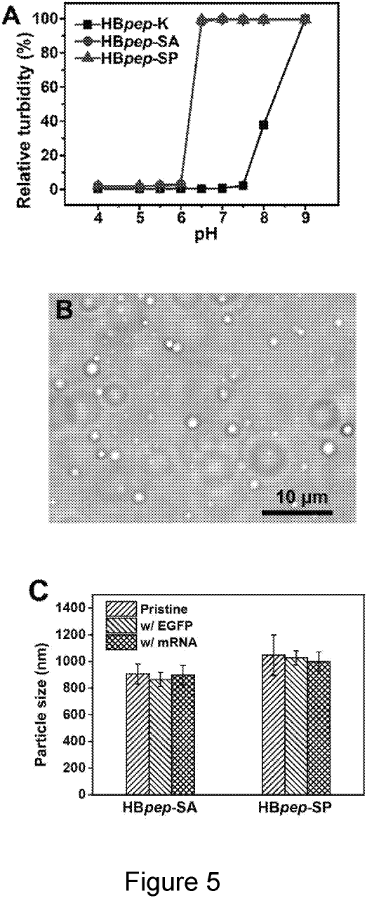

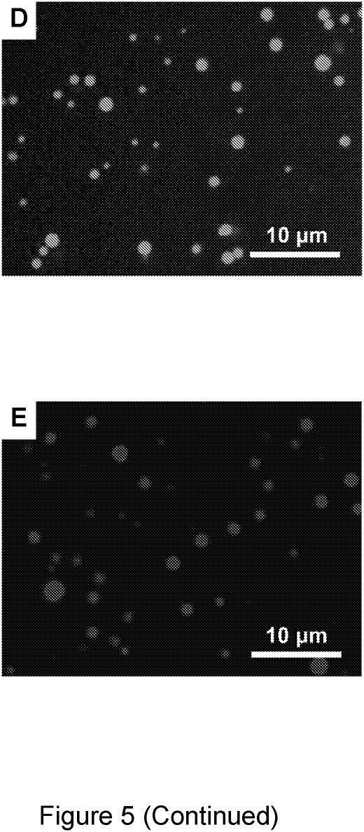

By single amino acid level manipulation, the pH range at which HBpep phase separates can be dramatically altered. The insertion of a single lysine at position sixteen (HBpep-K) could only phase separate at a higher pH of 9.0 (FIG. 5A), compared to the original HBpep that phase separates at ~ pH7.5 (Lim, Z.W. et al., Bioconjugate Chem., 2018, 29, 2176), suggesting that at a single amino acid level manipulation, the pH range at which HBpep phase separates can be dramatically altered. Next, a disulfide-containing self-immolative (SR) moiety was conjugated to the ε-amine group of the inserted lysine residue (K) to neutralize the extra positive charge and increase the hydrophobicity of the peptide (FIG. 1). The conjugated moiety is self-immolative and can be fully cleaved through a series of auto-catalytic reactions, starting by the reduction of the disulfide bond followed by side-group rearrangements, eventually restoring the amine group of the lysine residue (K) (FIG. 1; Riber, C.F. et al., Adv. Healthcare Mat., 2015, 4, 1887; Deng, Z. et al., Macromolecular Rapid Comms., 2020, 41, 1900531; Tang, L. et al., Nat. Biotech., 2018, 36, 707). After the modification, both peptides with acetyl ended side chain (HBpep-SA) and phenyl ended side chain (HBpep-SP) were able to phase separate at the lower pH of 6.5 (FIG. 5A) and formed stable microdroplets at near-physiological conditions (FIG. 5B). This design allowed the modified peptides (HBpep-SR) to form coacervate microdroplets with a diameter of ca.1 µm (FIG. 5C). Critically, HBpep-SR peptides were able to recruit a wide range of macromolecules during the self-coacervation process at a pH of 6.5, such as EGFP (FIG. 5D) or fluorescently-labelled mRNA (FIG. 5E), and the cargo-loaded peptide coacervates were stable at near-physiological conditions until internalization by the cells.

### In Vitro Insulin Release in the Presence of DTT

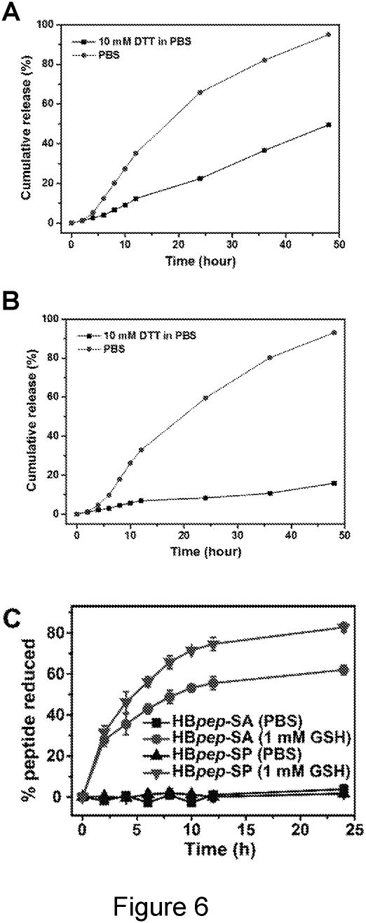

To evaluate the response of the peptide coacervates HBpep-SR in a reducing environment, FITC-insulin was released from the peptide coacervates in the presence or absence of DTT. In the presence of reducing agent DTT, the release rates of both peptide coacervates HBpep-SA (FIG. 6A) and HBpep-SP (FIG. 6B) increased significantly, suggesting that the peptide coacervates HBpep-SR can be disassembled in the presence of a reducing agent and can simultaneously release the cargo, insulin.

### In Vitro Macromolecule Release in the Presence of GSH

Similarly, owing to the self-immolative nature of the flanking moiety (SR), GSH-triggered reduction caused the disassembly of the peptide coacervate microdroplets HBpep-SA and HBpep-SA which in turn, released the cargo directly in the cytosol (FIG. 6C). The inventors’ findings suggest that reducing agents such as GSH, which abundantly exists in the cytosol triggers the reduction and cleavage of the entire modified side-chain (SR), eventually converting HBpep-SR back to HBpep-K (FIG. 1). Further, since HBpep-K does not remain in the biphasic regime at neutral pH but reverts to the single phase (e.g. monomeric peptide in solution, FIG. 5A), GSH-triggered reduction caused the disassembly of HBpep-SR, in turn releasing the cargo directly in the solution. It is also noteworthy that a simple modification at the end of the flanking moiety of HBpep-SR (HBpep-SA vs HBpep-SP) resulted in significant variation in the rate of peptide reduction, which could be an added strategy to control the kinetics of therapeutic release (FIG. 6C).

### Example 2: EGFP and Insulin Model Intracellular Protein Delivery Mediated by Redox-Responsive Peptide Coacervates

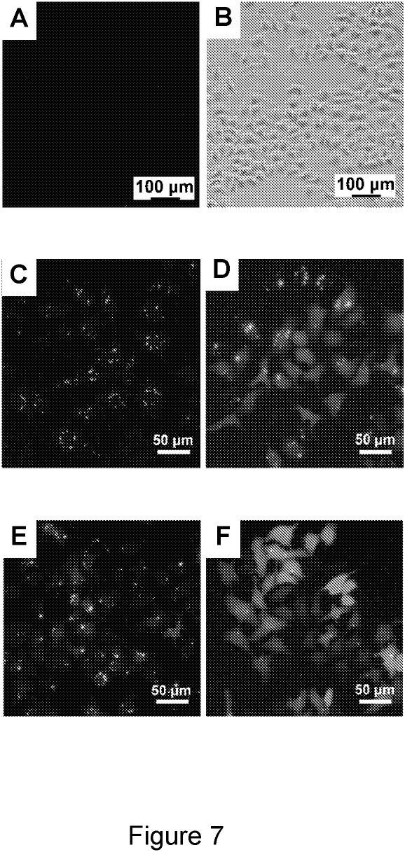

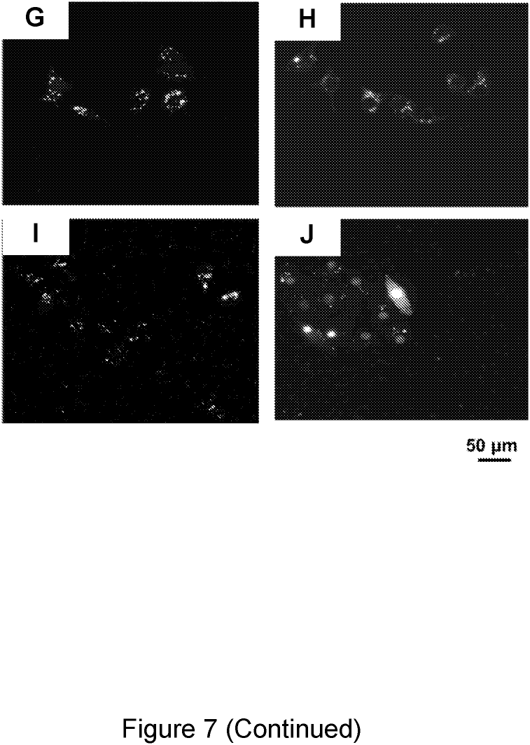

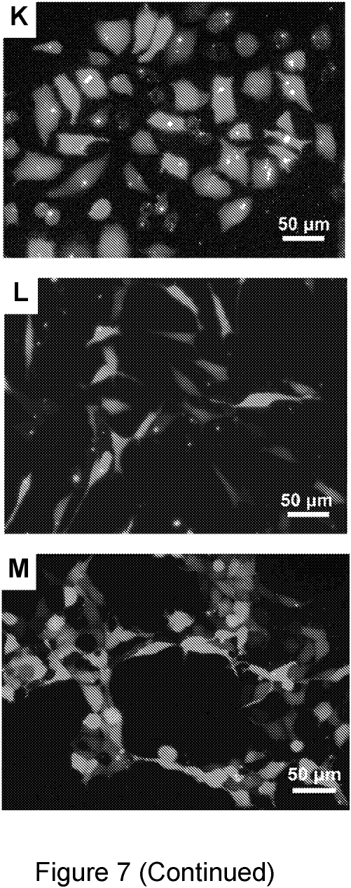

To evaluate the intracellular delivery efficiency of the peptide coacervates (HBpep-SR), EGFP was first employed as a model protein and recruited inside both HBpep-SA and HBpep-SP coacervates, before being incubated with liver cancer cells (HepG2). As a control, EGFP alone could not cross the cell membrane (FIGS. 7A, 7B). EGFP-loaded HBpep-SA peptide coacervates however were internalized by the cells within 4 hours (FIG. 7C), and subsequently released inside the cytoplasm within 24 hours (FIG. 7D). Similarly, EGFP-loaded HBpep-SP peptide coacervates were internalized by HepG2 cells within 4 hours (FIG. 7E), and subsequently released inside the cytoplasm within 24 hours (FIG. 7F). Similarly, insulin-loaded HBpep-SA and HBpep-SP coacervates were internalized by HepG2 cells within 4 hours (FIGS. 7G and 7I, respectively), and subsequently released insulin inside the cytoplasm within 24 hours (FIGS. 7H and 7J, respectively). Another finding was that HBpep-SP exhibited a faster release rate than HBpep-SA and started to deliver its EGFP cargo after 4 hours, which is consistent with the faster reduction rate of HBpep-SP (FIG. 6C). This further highlights the possibility of controlling the kinetics of cargo release by slight modifications of the conjugate moiety side group. To further investigate the versatility of this delivery system, EGFP loaded HBpep-SP peptide coacervates were further tested on another cancerous cell line (A549) as well as two healthy cell lines, namely NIH 3T3 and HEK 293. Based on the fluorescence signals observed inside the cells, the intracellular delivery and release ability of the HBpep-SP peptide coacervates was verified for A549 (FIG. 7K), NIH 3T3 (FIG. 7L) and HEK 293 (FIG. 7M) cell lines.

### Example 3: Intracellular Delivery and Release of Proteins With Different MWs and IEPs By HBpep-SP Peptide Coacervates

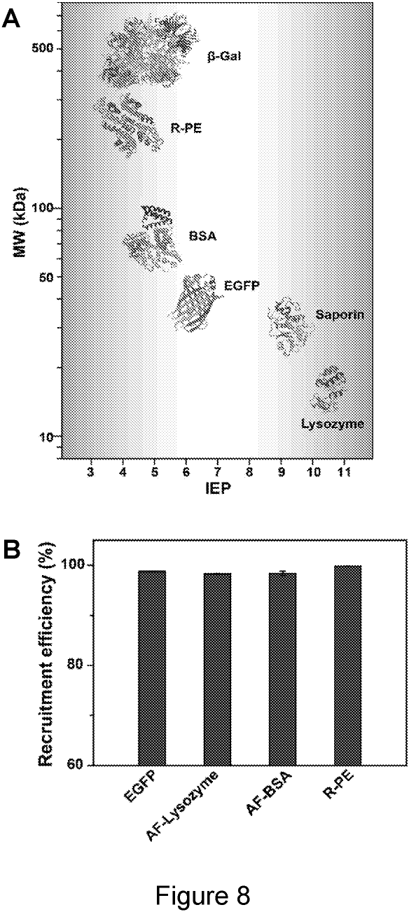

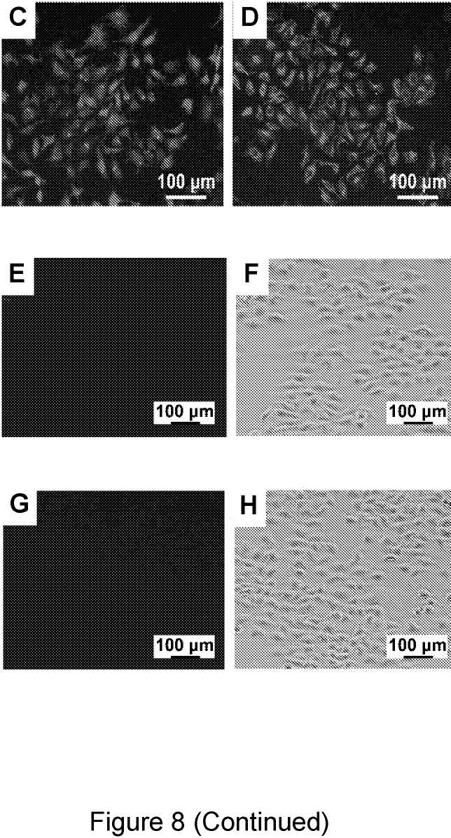

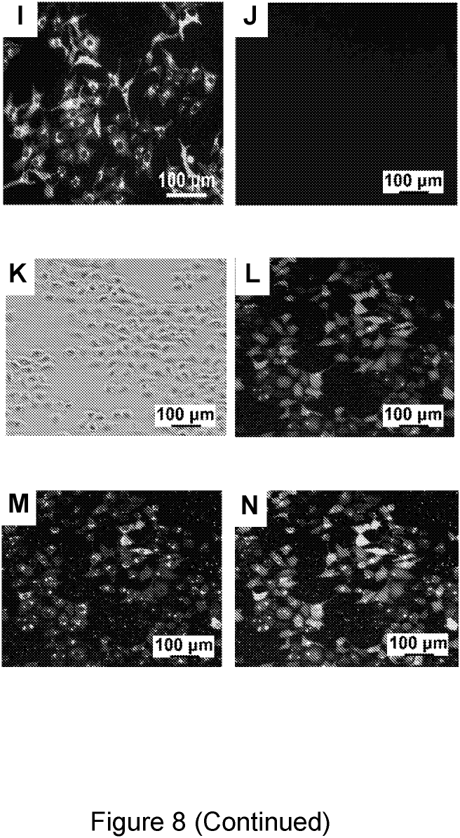

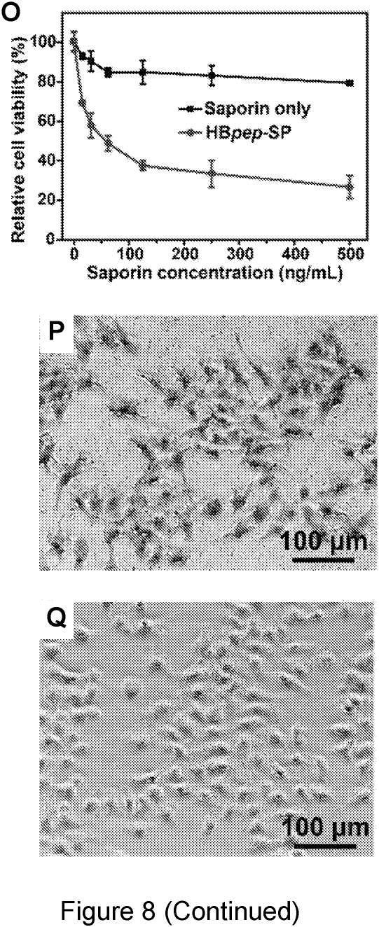

After the successful delivery and release of EGFP, the inventors assessed if proteins with a wide range of MWs and isoelectric points (IEPs) could also be delivered into HepG2 cells using HBpep-SP peptide coacervates (FIG. 8A). The inventors first assessed lysozyme and bovine serum albumin (BSA), two common proteins with significantly different MWs and IEPs. HBpep-SP peptide coacervates (1 mg/mL) was found to effectively recruit proteins of varying MWs and IEPs, including EGFP, Alexa Fluor 488 (AF)-labeled lysozyme, AF-BSA and AF-R-PE at a concentration of 0.1 mg/mL (FIG. 8B). It was also found that lysozyme (MW = 14.5 kDa; FIG. 8C) and BSA (MW = 66.5 kDa; FIG. 8D), two common proteins with significantly different MWs and IEPs could be delivered into HepG2 cells and released into the cytoplasm within 24 hours. On the other hand, in their free form (not recruited in the HBpep-SP peptide coacervates), neither lysozyme (FIGS. 8E, 8F) nor BSA (FIGS. 8G, 8H) were internalized by HepG2 cells.

To further challenge the MW ceiling of the cargo proteins, R-PE, a larger red fluorescence protein (MW = 255 kDa) was effectively recruited inside the HBpep-SP peptide coacervates (FIG. 8B), and incubated with HepG2 cells. After 4 hours of uptake and another 20 hours of release, a strong red fluorescence signal was detected inside the cytoplasm, confirming that R-PE was delivered and released inside HepG2 cells (FIG. 8I). Conversely, R-PE in its free form was not internalized by HepG2 cells (FIGS. 8J, 8K). Intracellular co-delivery of both EFGP and R-PE in HBpep-SP peptide coacervates was next tested. Both green (EGFP; FIG. 8L) and red (R-PE; FIG. 8M) fluorescence signals were observed in HepG2 cells treated with EGFP / R-PE co-loaded HBpep-SP peptide coacervates (EGFP / R-PE; FIG. 8N), demonstrating the ability of the HBpep-SP peptide coacervate system to synergistically deliver a combination of protein therapeutics.

Beside the successful delivery and release of cargo proteins, maintaining their bioactivity after delivery is critical for protein-based therapies. Saporin from Saponaria officinalis seeds is a well-known ribosome inactivating protein (Lv, J. et al., Biomat., 2018, 182, 167; Wang, M. et al., Angewandte Chemie Int. Ed., 2014, 53, 2893). But due to its poor membrane permeability, a suitable delivery system is required for further applications of saporin in biomedicine (Lv, J. et al., Biomat., 2018, 182, 167). As shown in FIG. 8O, the viability of HepG2 cells treated with saporin-loaded HBpep-SP peptide coacervates significantly decreased compared to those treated with saporin alone. This demonstrates not only that saporin was delivered and released from HBpep-SP peptide coacervates, but also that its bioactivity was preserved during the recruitment and delivery process.

To further confirm the versatility of the HBpep-SR peptide coacervate delivery system, β-Gal, a very high MW enzyme (MW = 430 kDa) was selected to be recruited into the HBpep-SP peptide coacervate. Intracellular delivery of β-Gal is challenging because of the difficulty in forming complexes with common nanocarriers owing to its high MW (Mitragotri, S. et al., Nat. Reviews Drug Disc., 2014, 13, 655). However, as shown in FIG. 8P, almost all of the HepG2 cells treated with β-Gal-loaded HBpep-SP coacervates turned blue due to the pigment generated by the β-Gal-catalyzed hydrolysis of the substrate 5-bromo-4-chloro-3-indolyl-p-D-galactoside (X-Gal). In contrast, there was no blue pigment formation in cells treated with β-Gal alone (FIG. 8Q), further corroborating that HBpep-SP peptide coacervates were capable of delivering large enzymes and maintain their activities.

Taken together, these results show that HBpep-SR peptide coacervates are capable of efficiently recruiting and directly delivering in the cytosol a wide range of proteins regardless of their MWs and IEPs, with a process of cargo recruitment that is fully aqueous, easy, and rapid. These characteristics enable HBpep-SR peptide coacervates to recruit both native as well as recombinant proteins without further chemical modifications and to preserve their bioactivity, making this approach a promising and flexible platform for single- and multi-protein based therapies.

### Example 4: Intracellular Peptide Mediated by HBpep-SP Peptide Coacervates

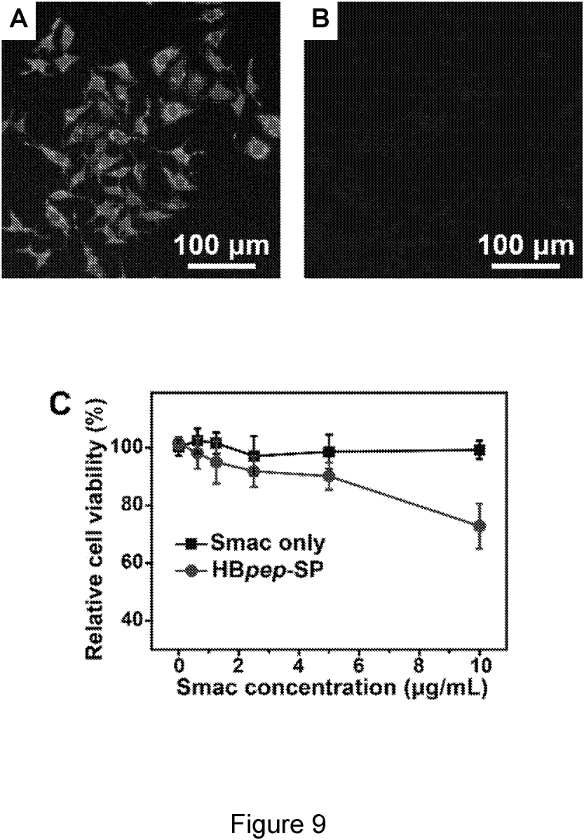

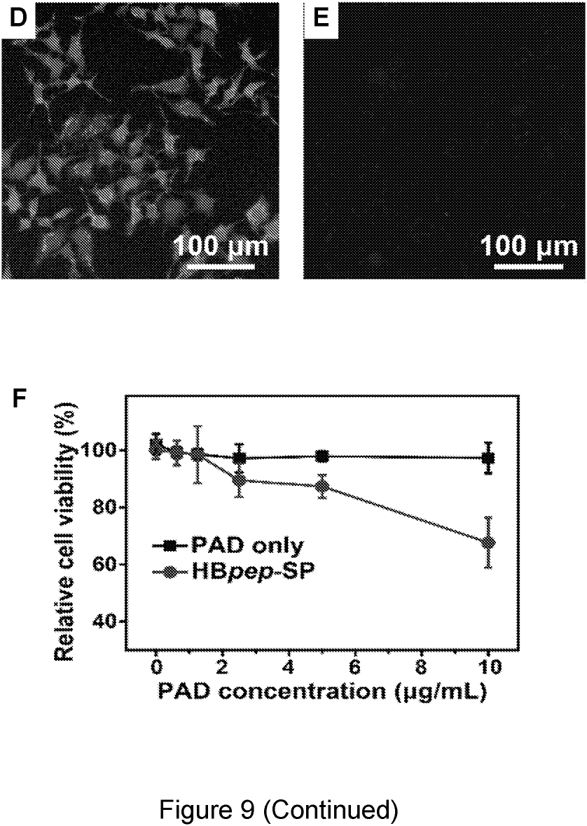

Compared to protein-based therapeutics, peptides display specific advantages such as a low immune response and scalability (Fosgerau, K. et al., Drug Disc. Today, 2015, 20, 122). Therefore, two short peptides including the second mitochondria-derived activator (Smac, AVPIAQK) and the proapoptotic domain (PAD, KLAKLAK KLAKLAK) peptides were selected to be delivered into HepG2 cells using HBpep-SP peptide coacervates. Both the Smac and PAD peptides have previously been demonstrated to exhibit anticancer effects by promoting caspase activity or causing mitochondrial membrane disruption (Li, M. et al., ACS Appl. Mat. & Interfaces, 2015, 7, 8005; Toyama, K., Bioconjugate Chem., 2018, 29, 2050). As shown in FIG. 9A, strong fluorescence signals were detected inside HepG2 cells treated with FITC-Smac loaded HBpep-SP peptide coacervates. In contrast, FITC-Smac alone could not cross the cell membrane (FIG. 9B). Similar results were also obtained in the delivery of FITC-PAD loaded HBpep-SP peptide coacervates (FIG. 9D). On the other hand, FITC-PAD alone was not able to cross the cell membrane (FIG. 9E). Furthermore, the anticancer activity of Smac and PAD loaded HBpep-SP peptide coacervates was evaluated as shown in FIGS. 9C and 9F, respectively. HepG2 cells treated with Smac-loaded and PAD-loaded HBpep-SP peptide coacervates showed 28% and 33% of cell death, respectively, at 10 µg/mL concentration. In comparison, there was negligible cytotoxicity for the cells treated with Smac or PAD alone (FIGS. 9C, 9F). These results indicate that the HBpep-SP peptide coacervate system can also deliver short therapeutic peptides.

### Example 5: mRNA Delivery Mediated by HBpep-SP Peptide Coacervates

Gene therapy has long been considered as a possible cure for serious diseases such as cancer, genetic disorder, and infectious diseases (Naldini, L., Nat., 2015, 526, 351). Among these, mRNA-based therapy has recently attracted increasing interest because of its biosafety and the ability for mass production (Pardi, N. et al., Nat., 2017, 543, 248; Pardi, N. et al., Nat. Comms., 2017, 8, 14630). In its most successful and dramatic application to date, mRNA-based technology ended up being the frontrunner for vaccine design against the COVID-19 pandemic (Chung, Y.H. et al., ACS Nano., 2020, 14, 12522). Therefore, it was further assessed if the redox-responsive HBpep-SR coacervate microdroplets could also be used to deliver mRNA.

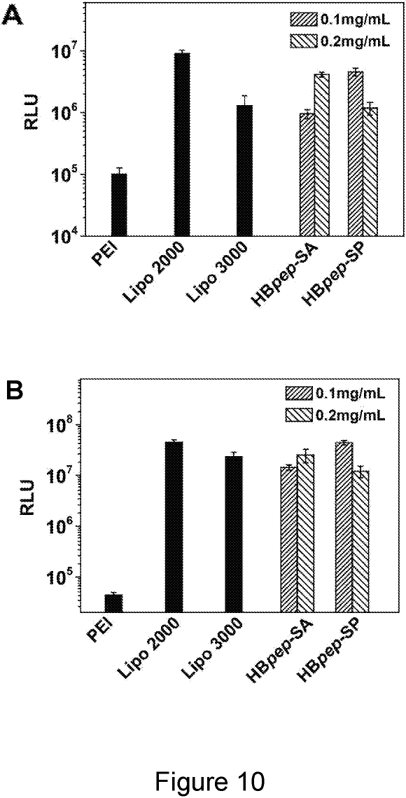

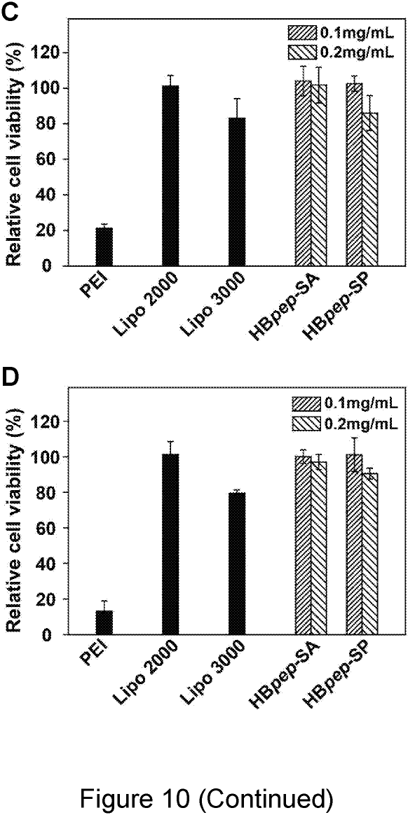

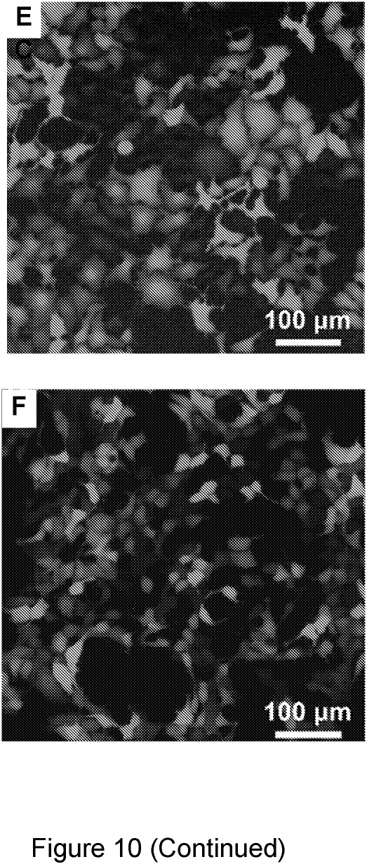

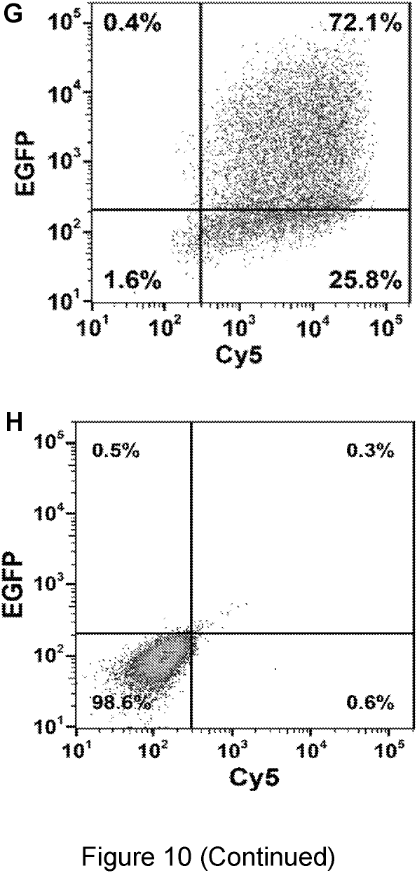

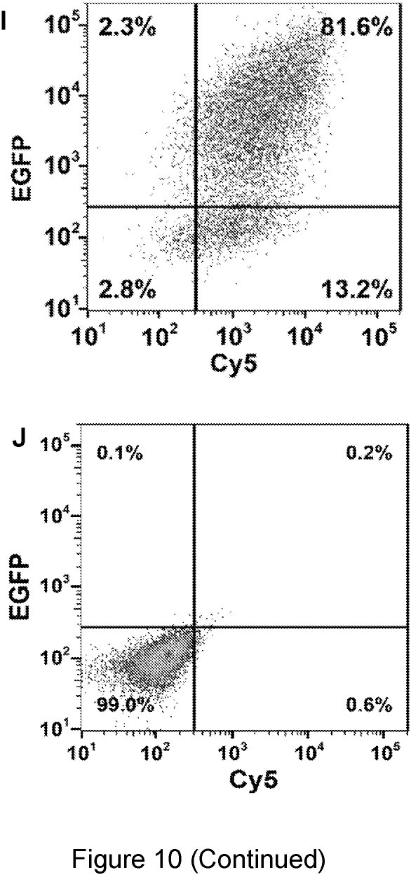

The transfection efficiency was evaluated using mRNA encoded with the reporter gene luciferase in both HepG2 and HEK293 cell lines. Three commonly-used transfection systems, including polyethylenimine (PEl), lipofectamine 2000 and 3000, were employed as control groups. As shown in FIG. 10A, at the optimal peptide concentration, the transfection efficiencies of HBpep-SA and HBpep-SP peptide coacervates were higher than PEl and lipofectamine 3000, but slightly lower than lipofectamine 2000 in HepG2 cells. On the other hand, in HEK293 cells, HBpep-SP peptide coacervates showed comparable transfection efficiency with lipofectamine 2000 (FIG. 10B). Importantly, neither HBpep-SA nor HBpep-SP peptide coacervates with luciferase-encoding mRNA caused cytotoxicity at their optimal concentration (FIGS. 10C and 10D, respectively).

After the successful delivery of luciferase-encoding mRNA, the transfection efficiency of HBpep-SP peptide coacervates was further investigated with EGFP-encoding mRNA labeled with Cy5 dye. Based on the fluorescence micrographs, the vast majority of HepG2 (FIG. 10E) and HEK293 (FIG. 10F) cells were successfully transfected with mRNA as most cells exhibited intense green fluorescence. The transfection efficiency was then quantified using fluorescence-activated cell sorting (FACS) measurements. As shown in FIGS. 10G and 10H, the uptake efficiency of EGFP-encoding mRNA loaded in HBpep-SP peptide coacervates reached around 98% in HepG2 cells. Furthermore, 72% of HepG2 cells expressed EGFP after 24 hours.

For HEK293 cells, as shown in FIGS. 101 and 10J, 94.8% of cells exhibited coacervates internalization and 81.6% expressed EGFP after 24 hours. Such a high mRNA transfection efficiency suggests that the redox-responsive HBpep-SP peptide coacervates represent an efficient vector for gene therapy. Other nucleic acids such as plasmid DNA, microRNA and small interfering RNA could in principle be delivered using this platform. In combination with their protein delivery ability, HBpep-SP peptide coacervates may also be employed as a tool for the delivery of protein/nucleic acid complex, which is a critical step in genome editing systems such as CRISPR / Cas9 (Liu, C. et al., J. Controlled Release, 2017, 266, 17).

### Example 6: Internalization Mechanism Study of HBpep-SP Peptide Coacervates

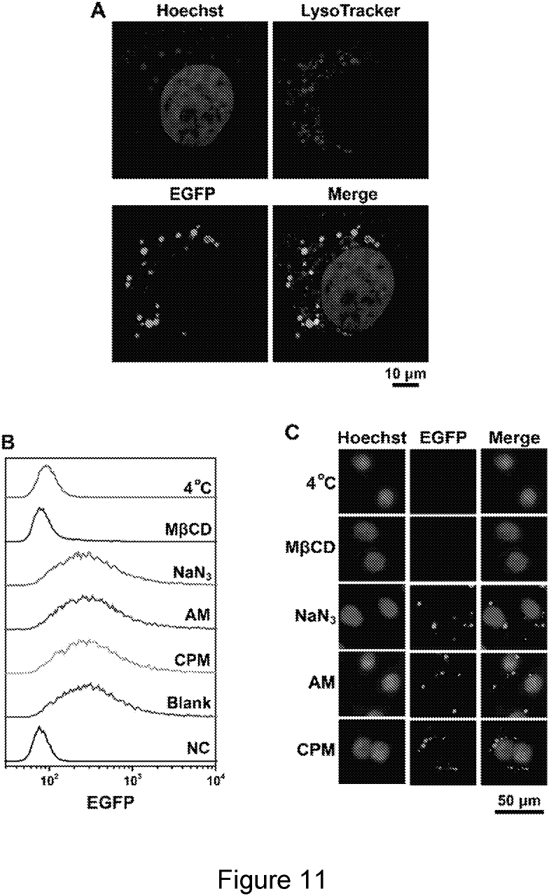

With a size of ca. 1 µm (FIG. 5C) — significantly larger than typical nanocarriers —and with liquid-like characteristics, it is intriguing that the peptide coacervate microdroplets display such a high cell uptake efficiency, which suggests a distinct internalization pathway than regular endocytosis. To verify if the HBpep-SP coacervates bypass endocytosis, the LysoTracker was used to stain acidic organelles such as lysosomes (Noack, A. et al., PNAS, 2018,115, E9590). Based on confocal microscopy images (FIG. 11A), EGFP-loaded HBpep-SP coacervates showed no colocalization with lysosomes. HepG2 cells were also treated with endocytosis inhibitors, including the clathrin-mediated endocytosis inhibitor chlorpromazine (CPM; Panja, P. et al., J. Phys. Chem. B, 2020, 124, 5323; Sangsuwan, R. et al., J. Am. Chem. Soc., 2019, 141, 2376), the pinocytosis inhibitor amiloride (AM; Panja, P. et al., J. Phys. Chem. B, 2020, 124, 5323; Lin, Q. et al., Pharma. Res., 2014, 31, 1438), and the energy-dependent endocytosis inhibitor sodium azide (NaN₃; Lin, Q. et al., Pharma. Res., 2014, 31, 1438; Xu, C. et al., Int. J. Pharma., 2015, 493, 172). None of these inhibitors significantly affected the uptake of EGFP-loaded HBpep-SP coacervates (FIGS. 11B, 11C).

However, HepG2 cells pre-treated with methyl-β-cyclodextrin (MβCD) showed almost no uptake of HBpep-SP peptide coacervates. The effect of MβCD is to deplete cholesterol (Mout, R. et al., ACS Nano., 2017, 11, 2452), which apparently blocked the internalization of HBpep-SP coacervates, suggesting that the mechanism of coacervates uptake is cholesterol-dependent lipid rafting (Panja, P. et al., J. Phys. Chem. B, 2020, 124, 5323). Low temperature treatment of the cells also resulted in a similar inhibition effect, which may be related to the lower fluidity of the membrane at low temperature (Murata, N. et al., Plant Physiol., 1997, 115, 875). These results nevertheless indicate that HBpep-SP peptide coacervates avoid endocytosis and endosomal escape, or cell membrane fusion — the two main mechanisms of intracellular delivery (Goswami, R. et al., Trends in Pharmacol. Sci., 2020, 41, 74) — such that the biomacromolecular cargos are directly delivered and released inside the cytosol, with a bioactivity that is also preserved.

In summary, it has been shown that HBpep conjugated with self-immolative (SR) moieties exhibit LLPS, forming coacervate microdroplets within which a wide range of biomacromolecules including proteins, peptides, and mRNA can be efficiently recruited. The cargo-loaded coacervates can be delivered into various cell lines and achieve redox-triggered cargo release directly in the cytosol. The versatility of cargo recruitment and release allows these redox-responsive coacervates to deliver a single or a combination of macromolecular therapeutics, making this intracellular delivery platform a promising candidate for the treatment of cancer, metabolic, and infectious diseases. It is noteworthy that the approach does not involve either endosomal escape or cell membrane fusion (the two main mechanisms of intracellular delivery; Goswami, R. et al., Trends in Pharmacol. Sci., 2020, 41, 74) and that the coacervates are micron-size carriers as opposed to nanocarriers used in the vast majority of current intracellular delivery strategies. Presumably, the liquid-like properties of coacervates achieved via LLPS is critical in their ability to cross the cell membrane, resulting in a cholesterol-dependent uptake, although the precise entry mechanism is still unclear and currently under investigation.

The entire disclosure of each document cited (including patents, patent applications, journal articles, abstracts, laboratory manuals, books, or other disclosures) in the Background, Detailed Description, and Examples is hereby incorporated herein by reference.

It is to be understood that the disclosures are not limited to particular compositions or methods, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. Unless defined otherwise, all technical and scientific terms used herein have the same meaning as commonly understood by one of ordinary skill in the art to which the disclosure pertains. Although any methods and materials similar or equivalent to those described herein can be used in the practice for testing, specific examples of appropriate materials and methods are described herein.

A number of embodiments of the disclosure have been described. Nevertheless, it will be understood that various modifications may be made without departing from the spirit and scope of the present disclosure. Accordingly, other embodiments are within the scope of the following claims.

1. An isolated peptide comprising or consisting of the amino acid sequence,
(GHGXY)ₙ K (GHGXY)ₘ Z,
(GHGXY K)ₙ (GHGXY)ₘ Z, or
(GHGXY)ₙ (K GHGXY)ₘ Z, wherein
X is valine (V), leucine (L) or proline (P),
Z is tryptophan (W) or absent,
n is 0, 1, 2, 3, 4 or 5,
m is 0, 1, 2, 3, 4 or 5, and
n+m is 3, 4 or 5, preferably 5.

2. The isolated peptide of claim 1,
wherein the isolated peptide comprises or consists of an amino acid sequence selected from the group consisting of:
(i) K GHGXY GHGXY GHGXY GHGXY GHGXY W (SEQ ID NO: 1)
(ii) GHGXY K GHGXY GHGXY GHGXY GHGXY W (SEQ ID NO: 2)
(iii) GHGXY GHGXY K GHGXY GHGXY GHGXY W (SEQ ID NO: 3)
(iv) GHGXY GHGXY GHGXY K GHGXY GHGXY W (SEQ ID NO: 4)
(v) GHGXY GHGXY GHGXY GHGXY K GHGXY W (SEQ ID NO: 5)
(vi) GHGXY GHGXY GHGXY GHGXY GHGXY W K (SEQ ID NO: 6)
(vii) K GHGVY GHGVY GHGPY GHGPY GHGLY W (SEQ ID NO: 7)
(viii) GHGVY K GHGVY GHGPY GHGPY GHGLY W (SEQ ID NO: 8)
(ix) GHGVY GHGVY K GHGPY GHGPY GHGLY W (SEQ ID NO: 9)
(x) GHGVY GHGVY GHGPY K GHGPY GHGLY W (SEQ ID NO: 10)
(xi) GHGVY GHGVY GHGPY GHGPY K GHGLY W (SEQ ID NO: 11), or
(xii) GHGVY GHGVY GHGPY GHGPY GHGLY W K (SEQ ID NO: 12).

3. The isolated peptide of claim 1,
wherein the lysine residue (K) is modified at an epsilon (ε)- amino group with a self-immolative moiety.

4. The isolated peptide of claim 3,
wherein the self-immolative moiety comprises a disulfide (—S—S—) moiety.

5. The isolated peptide of claim 3,
wherein the self-immolative moiety has the formula —C(═O)—O—(CH₂)ₙ—S—S—R,
wherein n is 1, 2, 3, 4, or 5, and
wherein R is selected from: substituted or unsubstituted alkyl, alkenyl, cycloalk(en)yl, and aryl.

6. The isolated peptide of claim 5,
wherein R is —(CH₂)ₙ—O—C(═O)—R′,
wherein n is 1, 2, 3, 4, or 5, and
wherein R′ is selected from: C₁₋₄ alkyl, C₆-aryl, preferably phenyl, optionally substituted with halogen.

7. A composition for delivery of an active agent, the composition comprising a peptide coacervate,
wherein the peptide coacervate comprises:
(i) one or more isolated peptides according to claim 1; and
(ii) an active agent recruited in the peptide coacervate.

8. The composition of claim 7,
wherein the self-immolative moiety autocatalytically cleaves itself upon exposure to specific conditions selected from the group consisting of: pH changes, redox changes, exposure to release agents, and combinations thereof.

9. The composition of claim 7,
wherein the active agent is selected from the group comprising: proteins, (poly)peptides, carbohydrates, nucleic acids, lipids, (small) chemical compounds, nanoparticles, and combinations thereof.

10. The composition of claim 7,
wherein the active agent is a pharmaceutical or diagnostic agent.

11. The composition of claim 9,
wherein the active agent is a protein or (poly)peptide.

12. The composition of claim 11, wherein the protein or polypeptide is an antibody, antibody variant, antibody fragment or peptide.

13. The composition of claim 7,
wherein the composition is a pharmaceutical or diagnostic formulation for administration to a subject.

14. The composition of claim 7,
wherein the pH of the composition is > 5.0 and < 8.0.

15. A method for the recruitment of an active agent in a peptide coacervate, the method comprising:
(i) providing an aqueous solution of coacervate-forming peptides, wherein the coacervate-forming peptides are selected from the isolated peptides of claim 1;
(ii) combining the aqueous solution of the coacervate-forming peptides with an aqueous solution of an active agent; and
(iii) inducing coacervate formation.

16. The method of claim 15,
wherein the aqueous solution of the active agent is buffered such that the combination of the aqueous solution of the active agent with the aqueous solution of the coacervate-forming peptides has a pH of > 5.0 and < 8.0.

17. The method of claim 15,
wherein a volume ratio of the aqueous solution of the coacervate-forming peptides to the aqueous solution of the active agent is between 1 : 5 and 1 : 20.

18. A method for the delivery of an active agent, the method comprising:
(i) providing a composition comprising a peptide coacervate, wherein the peptide coacervate comprises:
a. one or more isolated peptides selected from the peptides of claim 3,
b. an active agent, wherein the active agent is recruited in the peptide coacervate, and
(ii) exposing the peptide coacervate to conditions that trigger the release of the active agent from the peptide coacervate.

19. A method for treating or diagnosing a condition or disease in a subject in need thereof, comprising:
(i) administering a composition comprising a peptide coacervate to a subject, wherein the peptide coacervate comprises:
a. one or more isolated peptides selected from the peptides of claim 3,
b. a pharmaceutical or diagnostic agent, wherein the pharmaceutical or diagnostic agent is recruited in the peptide coacervate, and
(ii) exposing the peptide coacervate to conditions that trigger the release of the pharmaceutical or diagnostic agent from the peptide coacervate.

20. The method of claim 19,
wherein the subject is a human afflicted by cancer,
wherein the pharmaceutical or diagnostic agent is an anti-cancer agent, and
wherein release is facilitated by the exposure of the peptide coacervate to a reducing environment, preferably glutathione (GSH), resulting in the reduction of the disulfide bond of the peptide coacervate.


SEQUENCE LISTING (12 sequences)

SEQ ID NO: 1 - protein, 27 aa, Artificial
  HBpep derivative
  MISC_FEATURE (5)..(5): Xaa can be Val, Leu or Pro
  MISC_FEATURE (10)..(10): Xaa can be Val, Leu or Pro
  MISC_FEATURE (15)..(15): Xaa can be Val, Leu or Pro
  MISC_FEATURE (20)..(20): Xaa can be Val, Leu or Pro
  MISC_FEATURE (25)..(25): Xaa can be Val, Leu or Pro
KGHGXYGHGXYGHGXYGHGXYGHGXYW

SEQ ID NO: 2 - protein, 27 aa, Artificial
  HBpep derivative
  MISC_FEATURE (4)..(4): Xaa can be Val, Leu or Pro
  MISC_FEATURE (10)..(10): Xaa can be Val, Leu or Pro
  MISC_FEATURE (15)..(15): Xaa can be Val, Leu or Pro
  MISC_FEATURE (20)..(20): Xaa can be Val, Leu or Pro
  MISC_FEATURE (25)..(25): Xaa can be Val, Leu or Pro
GHGXYKGHGXYGHGXYGHGXYGHGXYW

SEQ ID NO: 3 - protein, 27 aa, Artificial
  HBpep derivative
  MISC_FEATURE (4)..(4): Xaa can be Val, Leu or Pro
  MISC_FEATURE (9)..(9): Xaa can be Val, Leu or Pro
  MISC_FEATURE (15)..(15): Xaa can be Val, Leu or Pro
  MISC_FEATURE (20)..(20): Xaa can be Val, Leu or Pro
  MISC_FEATURE (25)..(25): Xaa can be Val, Leu or Pro
GHGXYGHGXYKGHGXYGHGXYGHGXYW

SEQ ID NO: 4 - protein, 27 aa, Artificial
  HBpep derivative
  MISC_FEATURE (4)..(4): Xaa can be Val, Leu or Pro
  MISC_FEATURE (9)..(9): Xaa can be Val, Leu or Pro
  MISC_FEATURE (14)..(14): Xaa can be Val, Leu or Pro
  MISC_FEATURE (20)..(20): Xaa can be Val, Leu or Pro
  MISC_FEATURE (25)..(25): Xaa can be Val, Leu or Pro
GHGXYGHGXYGHGXYKGHGXYGHGXYW

SEQ ID NO: 5 - protein, 27 aa, Artificial
  HBpep derivative
  MISC_FEATURE (4)..(4): Xaa can be Val, Leu or Pro
  MISC_FEATURE (9)..(9): Xaa can be Val, Leu or Pro
  MISC_FEATURE (14)..(14): Xaa can be Val, Leu or Pro
  MISC_FEATURE (19)..(19): Xaa can be Val, Leu or Pro
  MISC_FEATURE (25)..(25): Xaa can be Val, Leu or Pro
GHGXYGHGXYGHGXYGHGXYKGHGXYW

SEQ ID NO: 6 - protein, 27 aa, Artificial
  HBpep derivative
  MISC_FEATURE (4)..(4): Xaa can be Val, Leu or Pro
  MISC_FEATURE (9)..(9): Xaa can be Val, Leu or Pro
  MISC_FEATURE (14)..(14): Xaa can be Val, Leu or Pro
  MISC_FEATURE (19)..(19): Xaa can be Val, Leu or Pro
  MISC_FEATURE (24)..(24): Xaa can be Val, Leu or Pro
GHGXYGHGXYGHGXYGHGXYGHGXYWK

SEQ ID NO: 7 - protein, 27 aa, Artificial
  HBpep derivative
KGHGVYGHGVYGHGPYGHGPYGHGLYW

SEQ ID NO: 8 - protein, 27 aa, Artificial
  HBpep derivative
GHGVYKGHGVYGHGPYGHGPYGHGLYW

SEQ ID NO: 9 - protein, 27 aa, Artificial
  HBpep derivative
GHGVYGHGVYKGHGPYGHGPYGHGLYW

SEQ ID NO: 10 - protein, 27 aa, Artificial
  HBpep derivative
GHGVYGHGVYGHGPYKGHGPYGHGLYW

SEQ ID NO: 11 - protein, 27 aa, Artificial
  HBpep derivative
GHGVYGHGVYGHGPYGHGPYKGHGLYW

SEQ ID NO: 12 - protein, 27 aa, Artificial
  HBpep derivative
GHGVYGHGVYGHGPYGHGPYGHGLYWK